(19) United States
(12) Patent Application Publication
(10) Pub. No.: US 2025/0255778 A1
(43) Pub. Date: Aug. 14, 2025

(54) SCAFFOLDS FOR SELECTIVE SCAVENGING OF STORAGE LESION FROM BIOLOGICAL MATERIAL AND METHODS THEREOF

(71) Applicant: INSTITUTE FOR STEM CELL SCIENCE AND REGENERATIVE MEDICINE, Bangalore (IN)

(72) Inventors: Praveen Kumar Vemula, Bangalore (IN); Manohar Mahato, New Delhi (IN); Subhashini Pandey, Delhi (IN); Preethem Srinath, Chennai (IN); Utkarsh Bhutani, Lucknow (IN)

(73) Assignee: INSTITUTE FOR STEM CELL SCIENCE AND REGENERATIVE MEDICINE, Bangalore (IN)

(21) Appl. No.: 18/847,696

(22) PCT Filed: Mar. 17, 2023
(86) PCT No.: PCT/IB2023/052628

§ 371 Date: Sep. 17, 2024

(30) Foreign Application Priority Data
Mar. 17, 2022 (IN) 202241014827

(51) Int. Cl. A61J 1/14

(52) U.S. Cl. CPC A61J 1/1468

(57) ABSTRACT

The present invention relates to scaffolds, combinations and compositions thereof, for selective scavenging of storage lesions from biological material, in particular from stored cells. The invention also relates to systems for selective scavenging of storage lesions from biological material such as stored blood cells. Further, the invention relates to methods for selective scavenging of storage lesions from biological material such as stored blood cells.

### FIELD OF THE INVENTION

This present invention relates to scaffolds, combinations and compositions thereof, for selective scavenging of storage lesions from biological material, in particular from stored cells. The invention also relates to systems for selective scavenging of storage lesions from biological material such as stored blood cells. Further, the invention relates to methods for selective scavenging of storage lesions from biological material such as stored blood cells.

### BACKGROUND OF THE INVENTION

Blood transfusion is one of the most common therapeutic practices in intensive care units (ICU), where about 50-70% of the patients are transfused with blood units at some point during their stay (Netzer, G. et al. Transfusion practice in the intensive care unit: a 10-year analysis. Transfusion 50, 2125-2134 (2010). Typical indications for blood transfusion include sickle cell crisis, anemia, and severe blood loss.

The crisis of stored cells has always been a critical problem, in particular of stored red blood cells (RBC) units. Despite shortage, for example, India discards 1 million blood units every year due to the deterioration in their quality throughout 42 days (D). One of the main reasons that can be cited for dumping this enormous volume of collected blood was the deterioration of blood quality during storage time of 35-42 days. Current US FDA regulations permit the storage of RBC units for up to 42 days. However, in conditions like Sickle cell anaemia, the average storage duration for units transfused per therapy is about two weeks. This two-week window is also permissible for transfusions post cardiac surgery.

The deterioration of blood quality has been attributed to the storage lesions consisting of Damage Associated Molecular Patterns (DAMPs) formation in the blood bags during storage. The residual leucocytes and erythrocytes undergo biochemical, morphological, and structural changes in RBCs during the storage-aging process that lead to the formation of these storage lesions. These biochemical changes result in 1.) haemolysis (with a concomitant increase in extracellular free-iron, heme), 2.) accumulation of shed bioactive proteins and lipids, 3.) Neutrophil extracellular trap (NETs). NETs correspond to extracellular DNA generated by neutrophils in RBC units, 4.) cell-free mitochondrial DNA (mtDNA), and 5.) RBC-derived microparticles or Microvesicles (M.P.s/M.V.s). The most studied DAMPs in this context are as follows: (i) extracellular free-iron and free-hemoglobin (Hb) generated due to lysis of RBCs, (ii) bioactive lipids such as polyunsaturated fatty acids (PUFAs), (iii) extracellular DNA, and (iv) nucleosomes generated by neutrophils. DAMPs often lead to transfusion-related complications like altered oxygen affinity, infections, allergic reactions, Transfusion-Related Acute Lung Injury (TRALI), and sepsis in severe cases. These are a potential source of post-transfusion sequelae in vulnerable populations. Previous investigations in cardiac research have reported an increase in severe complications and mortality when RBCs stored for >2 weeks were used for transfusion and these increased complications are attributed to the accumulation of DAMPs in stored blood over a period of time.

Based on storage-time dependent accumulation of DAMPs, stored human RBC units are classified as young RBCs or fresh RBCs (RBCs of <14-21 days of storage) and old RBCs (stored between 21 and 42 days). Transfusion of old stored whole blood consists of a pool of stored lesions that are primary mediators in the pathophysiology of fatal diseases like Transfusion-Related Acute Lung Injury (TRALI), deep vein thrombosis, and organ dysfunction. It has been long established that leucocytes associated with DAMPs in donor's blood are potential immune elicitors in recipients and are are implicated in transfusion-related complications. To reduce the accumulation of leucocytes associated DAMPs, RBCs are treated with leukoreduction filters prior to storage. Leucoreduction leads to reduced formation of DAMPs like NETs. However, this technology does not profess to eliminate all kinds of DAMPs, especially those contributed by erythrocytes.

Therefore, in alternative to the leukoreduction process, previous studies have focused on improving storage conditions by alternative cryopreservation protocols (Henkelman, S., Noorman, F., Badloc, J. F. & Lagerberg, J. W. Utilization and quality of cryopreserved red blood cells in transfusion medicine. Vox Sang. 108, 103-112 (2015); Chang, A. L. et al. Previous cryopreservation alters the natural history of the red blood cell storage lesion. Shock 46, 89-95 (2016)), anaerobic storage (Burns, J. M. et al. Deterioration of red blood cell mechanical properties is reduced in anaerobic storage. Blood Transfus. 14, 80-88 (2016); Dumont, L. J., D'Alessandro, A., Szczepiorkowski, Z. M. & Yoshida, T. CO2-dependent metabolic modulation in red blood cells stored under anaerobic conditions. Transfusion 56, 392-403 (2016)), and usage of additives/rejuvenation solutions (Hess, J. R. et al. Successful storage of RBCs for 10 weeks in a new additive solution. Transfusion 40, 1012-1016 (2000); Pallotta, V., Gevi, F., D'Alessandro, A. & Zolla, L. Storing red blood cells with vitamin C and N-acetylcysteine prevents oxidative stress related lesions: a metabolomics overview. Blood Transfus. 12, 376-387 (2014); 19. Vani, R. et al. Prospects of vitamin C as an additive in plasma of stored blood. Adv. Hematol. 2015, 961049 (2015)). Additionally, washing stored RBCs with saline (0.9% NaCl) to remove accumulated bioactive factors is another established strategy approved to use in transfusion medicine. (Schmidt, A., Refaai, M., Kirkley, S. & Blumberg, N. Proven and potential clinical benefits of washing red blood cells before transfusion: current perspectives. Int. J. Clin. Transfus. Med. 4, 79-88 (2016); Bennett-Guerrero, E. et al. Randomized study of washing 40-42 day stored red blood cells. Transfusion 54, 2544-2552 (2014)). Past investigations suggest that adding preservatives, storing RBCs in an alkaline hypotonic solution with antioxidants, or rendering anaerobic storage conditions improves storage time up to 10-15 days. (Hess, J. R. et al. Successful storage of RBCs for 10 weeks in a new additive solution. Transfusion 40, 1012-1016 (2000); Pallotta, V., Gevi, F., D'Alessandro, A. & Zolla, L. Storing red blood cells with vitamin C and N-acetylcysteine prevents oxidative stress related lesions: a metabolomics overview. Blood Transfus. 12, 376-387 (2014); 19. Vani, R. et al. Prospects of vitamin C as an additive in plasma of stored blood. Adv. Hematol. 2015, 961049 (2015)) Additionally, the leukoreduction process is expensive.

However, these techniques did not prevent RBCs from producing DAMPs during storage time or improve overall quality. Still, these efforts do not exclusively focus on removing RBC and WBCs associated lesions and improving blood quality or shelf life. There appears to be no practical approach in the field to remove all classes of storage lesions and is more economical.

Prior arts like U.S. Pat. No. 9,877,476B2, CN107847395A, U.S. Pat. No. 5,624,794A, WO1996029864A1, EP2608816A4, ES2793484T3, U.S. Pat. No. 9,067,004B2, U.S. Pat. No. 9,199,016B2, EP3268015A4, MX2018014530A focus on anaerobic storage of blood and/or deal with removal of oxygen from blood to enhance storage.

U.S. Pat. No. 6,150,085A relates to a novel additive solution useful for the storage of human RBCs under refrigerated conditions.

In another finding, the application of polycationic nanofibers aims to adsorb anionic compounds such as heparin or nucleic acid. However, there is a whole cocktail of charged lesions present in stored RBCs. It ranges from free-histones, heme, Fe²⁺ to DNA and bioactive lipids. Each of these components can cause a significant reduction in the quality of stored blood/cells. These efforts do not focus on removing RBC-related lesions and improving blood quality or shelf life.

POLYCATIONIC MICROFIBERS AND METHODS OF USING THE SAME, (https://patentimages.storage.googleapis.com/56/64/3f/40a8cb518019e5/WO2018119422A1.pd f) discloses polycationic microfibers that are capable of sequestering or clearing certain molecules, complexes, or supramolecular assemblies from fluids, including bodily fluids and biological samples.

US 2017/0037544 A1 relates to methods of making polycationic nanofibers by grafting cationic polymers onto electro-spun neutral nanofibers and polycationic nanofibers produced by the methods.

U.S. Pat. No. 10,603,417B2 provides a system and methodology for the preservation of red blood cells in which red blood cells are, e.g., oxygen and carbon dioxide depleted undergo treatment and are stored in an anaerobic environment to optimize preparation for transfusion.

Since no practical approach is reported in the field that removes all kinds of storage lesions, there exists the need for a technology to solve this unmet clinical need to combat transfusion-related complexities. It involves the electrostatic interaction of charged storage lesions with cationic and/or anionic or zwitterionic scaffolds, and scavenging DAMPs using such scaffolds.

### BRIEF DESCRIPTION OF THE ACCOMPANYING DRAWINGS

FIG. 1 illustrates the synthesis and fabrication of poly-Tau and poly-Acr into hemocompatible hydrophobic nano fibre scaffolds, wherein:

FIG. 1a shows a scheme illustrating the synthesis of anionic polymer (pTau) and cationic polymer (pAcr) from PMVEMA polymer containing 50% hydrophobic domain where taurine and acridine bromide comjugation are 25% and 11% respectively.

FIG. 1b shows Field Emission Scanning Electron Microscopy (FESEM) images of scaffolds depicting that the fibres are continuous, bead free and contact angles measurements confirm their hydrophobicity.

FIG. 1c shows that surface charge measurements confirm the cationic and anionic property of pTau and pAcr.

FIG. 1d shows FESEM images of fibres post treatment, wherein, either in the presence of RBCs or its supernatant, there was no adherence to scaffolds.

FIG. 1e confirms that hemolysis assay confirmed the biocompatibility of pTau and pAcr with RBCs with hemolysis (%) less than 3% (n=3)

FIG. 1A shows Hemocompatible charged nanofibrous sheets scavenge storage lesions to improve the quality of blood.

FIG. 1Aa shows that stored RBCs produce storage lesion or damage associated molecular patterns (DAMPs) such as DNA, nucleosomes, heme (Hb), iron, and polyunsaturated fatty acids (PUFAs). Transfusion of DAMPs containing RBCs could lead to transfusion-related complications, including systemic inflammation and organ injury. The presence of DAMPs progressively reduces the quality of stored RBCs and limits their shelf-life. All DAMPs consist of positive or negative charge entities. Therefore, hemocompatible polymers poly-acridine and poly-taurine composed of complementary cationic and anionic charges, respectively, were prepared. Using these polymers, charge-bearing electrospun nanofibrous sheets (Tau-NFS and Acr-NFS) were prepared to scavenge charged DAMPs from stored RBCs, which remarkably enhanced the quality and shelf-life of stored RBCs.

FIG. 2 shows that charged nanofiber scaffolds scavenges DAMPs from both old, stored RBCs and its supernatant.

FIG. 2a is a schematic representation of collecting, packaging and storing pRBCs from 3 volunteers (male and female) for 42 days and treating it with pTau and pAcr scaffolds.

FIGS. 2b, 2c, 2d show that a combination of pTau and pAcr scaffolds scavenged significant amount of extracellular DNA, nucleosomes and heme from 42 D (“D” refers to “day”) RBCs supernatant and packed RBCs. Area of 12 cm² per scaffold (total=24 cm²) was taken to clean 1 ml of supernatant and pRBCs (4 degree Celsius) that exhibited the charge-based interaction of negatively charged DAMPs (extracellular DNA, bioactive lipids) with pAcr and positively charged (free nucleosomes and Hb) with pTau.

FIG. 2e shows that the scavenging of bioactive lipids—Arachidonic Acid (AA), 5-Hydroxyeicosatetraenoic acid (5-HETE), 12-Hydroxyeicosatetraenoic acid (12-HETE), 15-Hydroxyeicosatetraenoic acid (15-HETE) produced in pRBCs proved the broad-spectrum cleaning activity of charged scaffolds. The data is presented as Mean±S.D. unless otherwise stated.

FIG. 3 shows that intermittent scavenging of DAMPs at the 21^{st} and 28^{th} day enhanced the RBC quality at the 42^{nd} day.

FIG. 3a is a schematic representation of intermediate cleaning of pRBCs units and evaluating production of DAMPs by the end of 42 D.

FIGS. 3b, 3c and 3d shows that intermediate cleaning on the 28^{th} day demonstrated improved health of RBCs owing to less production of extracellular DNA, heme and nucleosomes by the end of 42 D compared to 21^{st} day cleaning of DAMPs. 24 cm² scaffold was used (pTau and pAcr) for cleaning 1 ml RBCs unit at 4 degree Celsius (n=3 different volunteers including males and females).

FIG. 3e shows that intermediate cleaning on Day 21 and Day 28 significantly confirmed the decreased formation of bioactive lipids compared to the untreated group.

FIG. 4 shows that intermittent scaffold treatment maintained the structural integrity and osmotic fragility of RBCs.

FIG. 4a shows a schematic representation of scaffold treatment performed on 21^{st} and 38^{th} day of pRBCs and evaluating the RBC health and fragility through FESEM and osmotic fragility analysis.

FIG. 4b shows SEM images of OD samples that confirmed the presence of Discocytes (healthy) whereas 42 D RBCs were majorly dominated by sphero-echinocytes and echinocytes (unhealthy).

FIG. 4c shows that the intermittent treatment on 28^{th} day confirmed an increased number of discocytes compared to 21^{st} day treatment.

FIG. 4d shows that in between treatment on 21^{st} D and 28^{th} D exhibited 2.5 and3.5 fold increase population of discocytes in RBCs compared to without treatment group.

FIG. 4e shows that the kinetics of stored pRBCs with the storage time (0-42 D) revealed increase MCF values and a shift in curve towards right.

FIG. 4g shows that osmotic fragility test of 42 D pRBCs, treated on 21^{st} and 28^{th} day of storage showed more resistance to lysis compared to untreated RBCs.

FIG. 5 shows that intermittent scavenging of DAMPs from murine stored blood slow down the ageing and extends the shelf life of stored RBCs.

FIG. 5a shows a schematic representation of the packaging and cleaning of murine RBC with pTau and pAcr scaffolds.

FIG. 5b shows that scavenging on 5^{th} or 10^{th} day (single) or both (twice) reduce the production of free heme and free nucleosomes at 14 D.

FIGS. 5c and 5d shows that 17 D stored RBCs treated twice with scaffolds significantly reduces the phosphatidyl serine exposure in the outer leaflet of plasma membrane compared to 17 D untreated group. FIG. 5d shows that biotin labelled RBCs were constant till 14 D but started declining in the untreated group whereas, in treated sets the population was intact and healthy till 17 D, suggesting the good quality of RBCs and improved shelf life.

FIG. 6 shows that twice treated 17 D old murine RBCs had reduced dose-responsive proinflammatory cytokine responses and lead to decreased RBC clearance, tissue iron delivery, and circulating NTBI levels, compared with transfusions of untreated stored RBCs. All transfusion recipients were male/female mice C57BL/6 mice (8-12 weeks of age), bred in-house at inStem/NCBS animal house. The results are presented as mean (SD) except where specified.

FIG. 6a shows the representative images of spleen obtained from mice 2 hours post transfusion with treated RBCs and untreated RBCs. Aliquotes (200 uL) of 14 D and 17 D old stored treated and non-treated RBCs (n=3) were transfused. Total iron was measured in spleens obtained after 2 hours post transfusion; the iron levels shown are compared with levels measured in control, transfused with fresh RBCs (n=3). The results are combined from 3 separate experiments.

FIG. 6b shows that C57BL/6 mice (n=3) transfused with untreated 14 D and 17 D stored RBCs or treated were sacrificed 2 hours post transfusion, and plasma cytokine levels were measured (as labeled).

FIG. 6c shows a representative image of old stored RBCs transfusion and its recovery. Transfusions of 14 D and 17 D stored treated and non-treated biotin labelled RBCs (24-h storage; n=3) were transfused (200 uL) and survival of transfused RBCs was calculated by streptavidin APC label flow cytometric tracking at 1 hour and 24 hours post transfusion.

FIG. 7 shows NMR findings substantiating ˜50 and 25% substitution of tetradecyl amine and taurine in pTau, while pAcr constituted ˜50% of tetradecyl amine and ˜11% of Acr-hexyl bromide substitution over PMVEMA.

FIG. 8A shows the significant increase in different classes of DAMPs production during the storage of Non-Leukoreduced (NLR) RBCs unit. The NLR units recorded a significant increase in extracellular DNA from 21 D to 28 D. The overall spike of ˜ 150 folds was observed from 0 D to 42 D in NLR blood, which completely complimented the level of other DAMPs. The free Hb increased up to 40 folds (˜ 0.03 g/dl to ˜ 0.8 g/dl) from 0 D to 42 D. All four non-polar lipids were quantified through LCMS, which showed a significantly elevated level from 28 D to 42 D of RBC storage. a-f Storage-induced production of DAMPs as a function of time. RBCs stored at 4° C. progressively produce DNA (a), free hemoglobin (Hb, b), and polyunsaturated fatty acids (PUFAs) such as AA (c), 5-HETE (d), 12-HETE (e), and 15-HETE (f).

FIG. 8B provides a comprehensive insight about the DAMPs production such as free Hb and arachidonic acid in leukoreduced (LR) RBC units with respect to time. Leukoreduced RBCs (LR units) showed a gradual increase in the production of extracellular DNA from 0 D to 42 D but compared to NLR, it was less by 30 folds. The increase in production on 42 D was approximately two folds higher than 28 D, affirming the formation of lesions associated with RBCs. A critical trend has been witnessed in free hemoglobin (Hb) production, which is increased up to 10 folds giving an idea that leukofilters had no control over the accumulation of RBC-associated DAMPs.

FIG. 9 indicates that scaffold optimization with respect to area and time shows pTau and pAcr scaffold in the combination of area 24 cm² is sufficient to scavenge maximum amount of DAMPs in 5 minutes. (A) Extracellular DNA and free Hb in 42 D NLR stored blood supt was selected as a marker to optimize the incubation time (5 min-30 min) with individual scaffolds (pTau and pAcr). This parameter was considered to scavenge the maximum DAMPs at 4° C. (without breaking the cold chain). Using cationic (pAcr) and anionic (pTau) scaffolds in combination for later scavenging experiments was based on the presence of a negative and positive charged lesion in pRBCs. The data suggested that 24 cm² of an individual scaffold could decrease extracellular DNA and Hb within 5 minutes from 1 ml of the supernatent. (B) In the area optimization, a gradual increase in surface area (>8 cm²) with an incubation time of 5 mins had no significant effect on the scavenging of extracellular DNA by pTau and pAcr scaffold groups.

FIG. 10 shows that no leaching phenomena was observed in (pAcr) scaffold with the blood component treatment.

FIG. 11 illustrates that scaffolds' scavenging ability shows significant results in scavenging RBCs associated DAMPs. The effectiveness of scaffold, pTau and pAcr scaffolds of 12 cm² area each (total surface area 24 cm²) were taken in combination to treat both LR 42 D old supernatent. The cleaning of DAMPs with scaffolds was performed for 5 minutes at 40 degree celsius. In LR units, the reduction in free Hb was highly evident in the supernatent group after treatment, it reduced to 50%. A remarkable drop of AA value after scaffolds treatment provides a method to purify LR blood that contains soluble bioactive lipids generated from RBCs.

FIG. 12 shows the intermittent scavenging of DAMPs on 28^{th}D treatment during storage enhanced LR RBC health at 42 D. The production of extracellular DNA in LR blood units was low as expected by the end of 42^{nd} D. Intermittent treatment on 21^{st} D and 28^{th} D treatment did not have any significant decrease in its concentration. 21^{st} D treatment did not impact the lysis rate of RBCs in LR units, whereas the 28^{th} D treatment limited the free Hb production by 50% compared to the untreated group. There was a significant production of AA, but intermittent treatment on 21^{st} D/28^{th} D drastically decreased its production by ten folds.

FIG. 13 shows that an increase in Mean Cell Fragility (MCF) values pushes the LR stored RBCs toward lysis: (A) LR units showed an increase in MCF values from approx. 0.35 to 0.5 stating the extent of RBCs health damage. (B) The 21^{st} and 28^{th} D intermittent treatment in LR units reduced the MCF values from the untreated group, shifting the hemolysis curve to the left side, i.e. the healthy side. The reduction in MCF values suggests that RBCs were healthier after intermediate treatment and are more resistant to lysis.

FIG. 14 shows that mice-stored RBCs also display a similar formation of DAMPs as in human blood. The data suggested a significant production of free Hb during 10 D-14 D of storage. The levels of free Hb doubled on the 14^{th} day compared to the 10^{th} day. A two-fold increase was also observed in the production of free nucleosomes. The production of DAMPs was estimated in the mice RBC units to identify the potential window for intermittent cleaning.

FIG. 15 shows that twice treated 17 D murine RBCs had reduced dose-responsive pro-inflammatory cytokine. Mice (age 8-12 weeks (males)) were infused with RBCs stored for 14 and 17 D respectively, with and without pTau and pAcr treatments. 2 hours post-transfusion, the mice were sacrificed and cytokine levels were measured in plasma. (A) Representation of cytokine array blots from all the groups. (B) In the 17 D untreated, the cytokine levels such as IL-16, TIMP-1, CXCL-10, IL-1F3, G-CSF were high, but they were not detected in the other treated groups showing reduced systemic inflammation after removing DAMPs.

FIG. 16 shows that the treatment group showed the recovery of stored labelled RBCs auto 17 days. Evaluating the enhanced shelf lives of treated RBCs, the biotin was transfused in both male and female mice (8-12 weeks). A few μL from different storage times was taken from the treated and untreated groups, and the percentage of the labelled RBCs was quantified using flow cytometry. After passing the shelf lives (14 days), treated RBCs were still in circulation compared to the untreated group. They were as healthy as fresh RBCs unit of age 0 D/7 D.

FIG. 17 shows freshly stored biotinylated RBCs were transfused into mice, collected blood at post-transfusion 1, 24, 48 and 72 hrs, and labelled with Streptavidin APCeFluor™, and quantified via flow cytometry. Flow cytometry profiles indicate that more than75% of labelled RBCs were in the circulation, which suggests the good quality of RBCs.

FIG. 18 shows a complete blood count of stored RBCs before and after Tau-AcrNFS treatment. a-e, Treatment of stored blood with Tau-AcrNFS did not cause loss of cells. Quantification of RBCs (a), WBCs (b), neutrophils (c), lymphocytes (d), and platelets (e) before and after treatment with Tau-AcrNFS, Data are mean±s.d. (n=3, from independent experiments). For a-e, P values were determined by two-tailed Student's t-test with Welch's correction using GraphPad PRISM 9, ns=not significant.

FIG. 19 shows Hypothermal storage of human RBCs induced DAMPs production and charged Tau-AcrNFS efficiently scavenged DAMPs from stored RBCs. a-f Storage-induced production of DAMPs as a function of time. RBCs stored at 4° C. progressively produce DNA (a), free hemoglobin (Hb, b), and polyunsaturated fatty acids (PUFAs) such as AA (c), 5-HETE (d), 12-HETE (e), and 15-HETE (f).g-m, Incubation of DAMPs accumulated 42 days-stored RBCs with anionic and cationic NFS, Tau-AcrNFS, for 5 min at 4° C. efficiently scavenged and reduced the concentration of DAMPs significantly. The relative concentration of DAMPs before and after Tau-AcrNFS treatment has significantly reduced accumulated DNA (g), nucleosomes (h), Hb (i), and PUFAs (j-m). Data are mean±s.d. (n=3, from independent experiments). For a-f, P values were determined by repeated measures one-way ANOVA, and for (g-m), by two-tailed Student's t-test with Welch's correction using Graph Pad PRISM 9, and exact P values are indicated. NS=not significant.

FIG. 20 shows scavenging of DAMPs using charged nanofibrous sheets. a,b. 42 days-stored RBCs were incubated with either Tau-NFS or Acr-NFS with varying surface area (8, 16, and 24 cm²). c, 42 days-stored RBCs were incubated with either Tau-NFS or Acr-NFS with varying incubation time (5, 15, and 30 mins). Data are mean±s.d. (n=3, from independent experiments); P values were determined by ordinary one-way ANOVA with Tukey's post hoc analysis using GraphPad PRISM 9, and exact P values are indicated.

FIG. 21 shows scheme for the synthesis of charged polymers, poly-acridine and poly-taurine. Poly-acridine and poly-taurine polymers were synthesized using solvent phase synthesis. The reaction conditions were as following; (i) anhydrous toluene, 120° C., 12 hours; (ii) tetradecyl amine, anhydrous tetrahydrofuran, 80° C., 3 hours; (iii) anhydrous tetrahydrofuran, 80° C., 12 hours; (iv) anhydrous dimethylformamide, 50° C., 16 hours; and (v) anhydrous tetrahydrofuran, 50° C., 16 hours. FIG. 22 shows synthesis of zwitterionic polymer from PMVEMA.

### SUMMARY OF THE INVENTION

The present invention provides a technology to solve an unmet clinical need for a technology to remove all classes of storage lesions from stored cells.

In one embodiment, the present invention provides nanofibre scaffolds for selective scavenging of storage lesion from stored cells comprising a backbone polymer functionalized with a group selected from: an anionic group, a cationic group, a combination of anionic group and cationic group, or a zwitterionic group.

In another embodiment, the present invention provides a system for selective scavenging of storage lesion from stored cells wherein the system comprises a nanofibre scaffold having a backbone polymer functionalized with an anionic group and a scaffold having a backbone polymer functionalized with a cationic group, wherein the storage lesion are selectively scavenged when stored cells are placed in contact with said scaffolds.

In yet another embodiment, the present invention provides a system for selective scavenging of storage lesion from stored cells wherein the system comprises a nanofibre scaffold having a backbone polymer functionalized with a zwitterionic group, wherein the storage lesion are selectively scavenged when stored cells are placed in contact with said scaffolds.

In an embodiment of the present invention the functionalized polymer in the scaffold and systems is in electrospun form.

In an embodiment of the present invention the backbone polymer is selected from the group comprising poly(acrylic acid), poly(4-vinylpyridine), Poly(4-vinylphenol), poly(allylamine), poly(vinyl alcohol), poly(methyl vinyl ether-alt-maleic anhydride) (PMVEMA), poly(vinyl chloride), poly(glycerol), poly(2-hydroxyethyl-methacrylamide), poly(2-((allyloxy)methyl) oxirane), poly(vinylpyrrolidone), poly(4-vinylbenzyl chloride-alt-maleic anhydride) and poly(vinylimidazole). The backbone polymer is preferably poly (methoxy vinyl ether-alt-maleic anhydride (PMVEMA).

In an embodiment, the present invention the backbone polymer is functionalized with an anionic group selected from 1,3-propane sultone, taurine, 1,3-dioxolan-2-one, 1,2-oxathiane 2,2-dioxide, phosphorochloridic acid, (3-aminopropyl)phosphonic acid, Pentanoate, 2-(methylamino)ethyl hydrogen phosphate, pentyl hydrogen phosphate and 4-(butylamino)butyl sulphate, preferably taurine. Preferably, the polymer is functionalized with taurine present in an amount from 8 to 50%.

In an alternative embodiment of the present invention, the polymer is functionalized with a cationic group selected from ethidium bromide derivative, cyanine dye, YO-1, proflavine derivative, acridine orange, ethylene blue derivative, ellipticine derivative, phenanthridine derivative, quinolinium derivative, thalidomide derivative, naphthalimide derivatives and quinoline derivative, preferably acridine. Preferably, the polymer is functionalized with acridine present in in an amount from 3 to 35%.

In an embodiment of the present invention, the cationic polymer has intercalating and positive charge characteristics.

In an alternative embodiment of the present invention, the polymer is functionalized with a zwitterionic group to make a zwitterionic polymer. Zwitterionic group has both cationic and anionic groups in the same molecules. These zwitterionic polymers are prepared by functionalizing the backbone polymer with a zwitterionic group or a combination of cationic group and anionic group as described hereinabove.

In an embodiment of the present invention, the scaffold is in the form of a sheet, bead, bag, insert device, tube, and the like, preferably a nanofibrous sheet.

In an embodiment of the present invention, the stored cells are selected from red blood cells (RBCs), white blood cells (WBCs), platelets and stem cells, preferably red blood cells (RBCs).

In yet another embodiment, the present invention provides a method for treating stored cells wherein said stored cells are placed in contact with said scaffolds or systems.

In yet another embodiment, the present invention provides a method of scavenging damage-associated molecular patterns (DAMPs) from stored cells wherein said stored cells are treated with said scaffolds or systems.

In one another embodiment, the present invention provides a method of scavenging damage-associated molecular patterns (DAMPs) from stored cells wherein said stored cells are treated with said scaffolds or systems.

In one another embodiment, the present invention provides a method of enhancing quality and shelf-life of stored cells by scavenging DAMPs from stored cells wherein said stored cells are treated with said scaffolds or systems.

In one another embodiment, the present invention provides a method to enhance the cell membrane integrity of stored cells by scavenging DAMPs from stored cells wherein said stored cells are treated with said scaffolds or systems.

In an embodiment of the present invention, the DAMPs are extracellular DNA, histones, nucleosomes, cell-free hemoglobin, iron, proteins, and polyunsaturated fatty acids.

In one another embodiment, the present invention provides use of said scaffold for treating the stored cells, comprising passing the stored cells through said scaffold.

In one another embodiment, the present invention provides a kit comprising said scaffold for treating the stored cells, comprising passing the stored cells through said scaffold.

In an embodiment of the present invention, the stored cells are red blood cells (RBCs), white blood cells (WBCs), platelets, and stem cells, preferably red blood cells (RBCs). In one another embodiment, the present invention provides a method for preparing a nanofibre scaffold as claimed in any of claims 1, 4 to 13 comprising: preparing a solution of polymer functionalized with a group selected from an anionic group, cationic group or zwitterionic group; and electrospinning the solution to obtain a nanofibre scaffold.

In an embodiment of the present invention, the polymer is made hydrophobic before functionalization with a group selected from an anionic group, cationic group or zwitterionic group.

### DETAILED DESCRIPTION OF THE INVENTION

The present invention relates to scaffolds for selective scavenging of storage lesions from biological material, in particular from stored cells. Said scaffolds are anionic and/or cationic or combinations thereof or zwitterionic.

Further, the present invention also relates to methods for selective scavenging of storage lesions from biological material such as stored cells.

The present invention discloses a technology to solve an unmet clinical need to remove all classes of storage lesions from stored cells such as, but not limiting to RBCs, white blood cells (WBCs), platelets and stem cells.

The present invention relates to scaffolds, combinations and compositions thereof, for selective scavenging of storage lesion consisting of DAMPs from biological material, particularly from stored red blood cells. Further, the invention relates to methods for selective scavenging of storage lesion consisting of DAMPs from biological material such as stored red blood cells.

The present invention relates to nanofibre scaffold for selective scavenging of storage lesion from stored cells comprising a backbone polymer functionalized with a group selected from: an anionic group, a cationic group, a combination of anionic group and cationic group, or a zwitterionic group.

The present invention also relates to a system for selective scavenging of storage lesion from stored cells wherein the system comprises a nanofibre scaffold having a backbone polymer functionalized with an anionic group and a scaffold having a backbone polymer functionalized with a cationic group, wherein the storage lesion are selectively scavenged when stored cells are placed in contact with said scaffolds.

The present invention also relates to a system for selective scavenging of storage lesion from stored cells wherein the system comprises a nanofibre scaffold having a backbone polymer functionalized with a zwitterionic group, wherein the storage lesion are selectively scavenged when stored cells are placed in contact with said scaffolds.

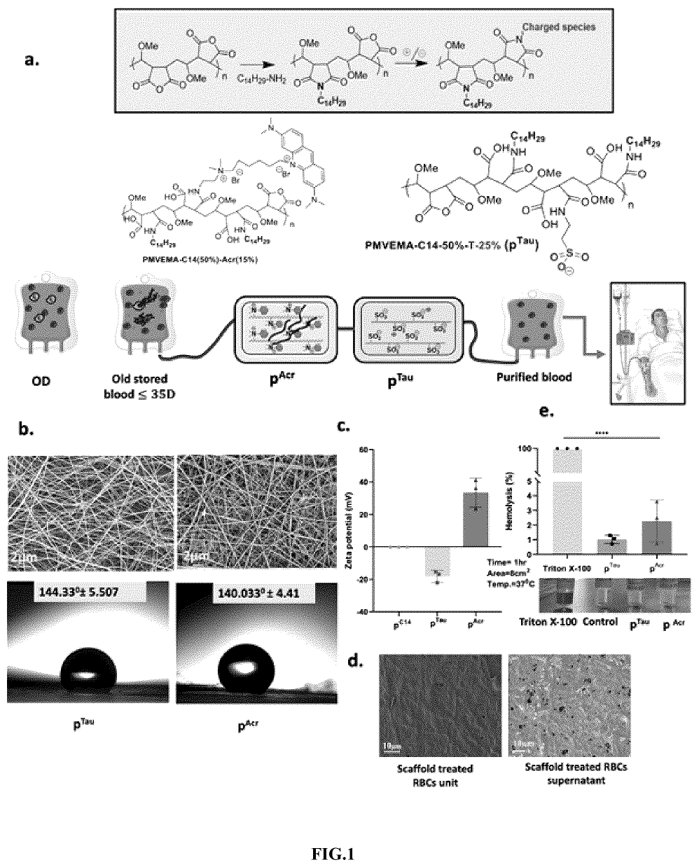

In an embodiment, the technology helps improve the overall quality of stored RBCs units to combat transfusion-related complexities and increase RBCs' shelf life to solve blood crisis problems. The present invention discloses an ideal user-compliant technology, easy-to-use, non-obstructive biocompatible scaffolds, combinations and compositions thereof, which could selectively scavenge the whole spectrum of DAMPs that are majorly cationic and anionic species. Said scaffolds are nanofibrous and these nanofibrous scaffolds selectively clean the lesions produced during the storage period to reduce the risk of transfusion-related complications in patients (FIG. 1a).

The present invention discloses a strategy for eliminating the whole spectrum of DAMPs by developing charged-nanofiber scaffolds for selective scavenging of cell-free extracellular DNA, histones, DNA-histones complex, cell-free haemoglobin, cell-free iron, proteins, and bioactive lipids from the old stored RBC units.

To scavenge neutrophil extracellular traps (NETs) and negatively charged bioactive lipids, nanofiber scaffolds from polymers fabricated with cationic moieties have been developed, that will possess intercalating and positive charge characteristics. Thus, such scaffolds could selectively bind to negatively charged DNA and lipids with high affinity.

In addition, to capture positively charged histones, in blood units, an anionic nanofiber scaffold has been developed from polymer consisting of sulphonic acids, which mimic the heparin sulphate glycoprotein (HSGP) that present on the cell surface.

The present invention investigates the efficiency of a selectively designed scaffold that could scavenge cationic and anionic DAMPs. The primary focus of this invention is to improve the quality and shelf life of stored blood by removing these storage lesions on a specific day during the storage window (42 days). This particular removal led to an overall low accumulation of DAMPs at the end of the 42^{nd} day of storage.

Scaffold development was based on identifying specific polymers, acting as backbone, which could be easily functionalized and modified to exhibit hydrophobic nature. Another requirement was that the polymers should not interact with the blood cells during the DAMPs cleaning process. The backbone polymers were selected from a group of polymers tabulated herein below:

| S. No. | Name | Structure |
|---|---|---|
| 1 | Poly(acrylic acid) | 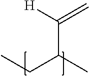 |
| 2 | Poly(4-vinylpyridine) | 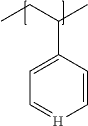 |
| 3 | Poly(4-vinylphenol) | 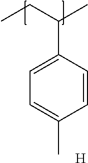 |
| 4 | Poly(allylamine) | 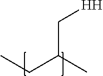 |
| 5 | Poly(vinyl alcohol) | 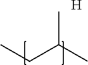 |
| 6 | Poly(methyl vinyl ether-alt-maleic anhydride) P(MVE-MA) | 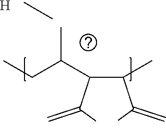 |
| 7 | Poly(vinyl chloride) | 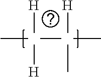 |
| 8 | Poly(glycerol) | 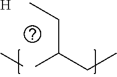 |
| 9 | Poly(2-hydroxyethyl- methacrylamide) | 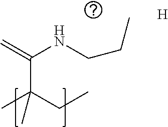 |
| 10 | Poly(2-((allyloxy) methyl)oxirane) | 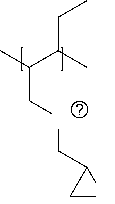 |
| 11 | Poly(vinylpyrrolidone) | 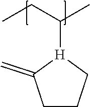 |
| 12 | Poly(4-vinylbenzyl chloride-alt- maleic anhydride) | 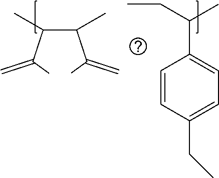 |
| 13 | Poly(vinylimidazole) | 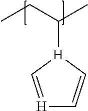 |

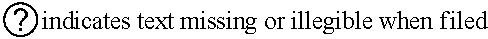

In a preferred embodiment, the high molecular weight poly methoxy vinyl ether-alt-maleic anhydride (PMVEMA) polymer was selected as the backbone polymer, and its hydrophobicity was enhanced by conjugation of alkyl amine with varied number of hydrocarbon chain, for example, tetra decyl amine. For the same it was mixed with solvent, selected from tetrahydrofuran (THF) and stirred at 50 to 80° C. until dissolution and then tetra decyl amine was added in 1:1 to 2:1 ratio and it was further stirred at 50 to 80° C. for about 3 to 16 hours and the solvent was removed to obtain the hydrophobic polymer in a powder form.

Further, the backbone polymer was functionalized with an anionic moiety and/or a cationic moiety. The reaction conditions will vary depending on the backbone polymer and compounds used for anionic and cationic functionalization used.

For anionic functionalization, the backbone polymer is mixed with an anionic compound selected from a group of compounds tabulated herein below:

| S. No. | Name | Structure |
|---|---|---|
| 1 | 1,3-propane sultone | 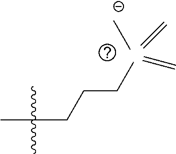 |
| 2 | Taurine | 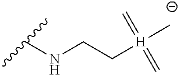 |
| 3 | 1,3-dioxolan-2-one | 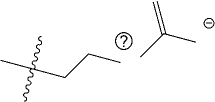 |
| 4 | 1,2-oxathiane 2,2-dioxide | 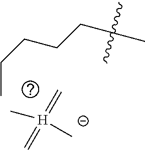 |
| 5 | phosphorochloridic acid | 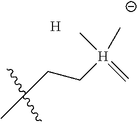 |
| 6 | (3-aminopropyl) phosphonic acid | 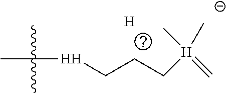 |
| 7 | Pentanoate | 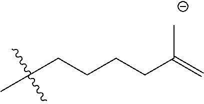 [SMILES: *CCCCC(=C)C] |
| 8 | 2-(methylamino) ethyl hydrogen phosphate | 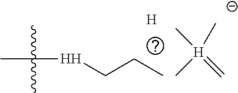 |
| 9 | pentyl hydrogen phosphate | 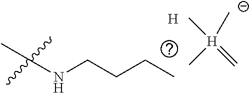 |
| 10 | 4-(butylamino) butyl sulfate | 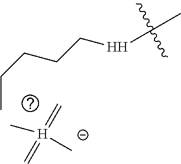 |

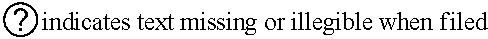

For cationic functionalization, the backbone polymer is mixed with a cationic compound selected from a group of compounds tabulated herein below:

| S. No. | Chemcial Name | Structure |
|---|---|---|
| 1 | Ethidium Bromide derivative | 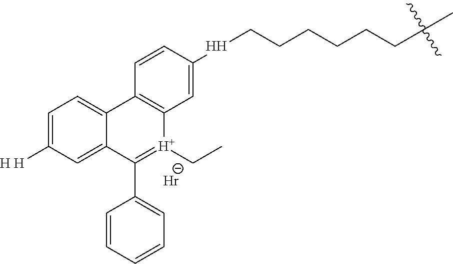 |
| 2 | The cyanine dye, YO-1, | 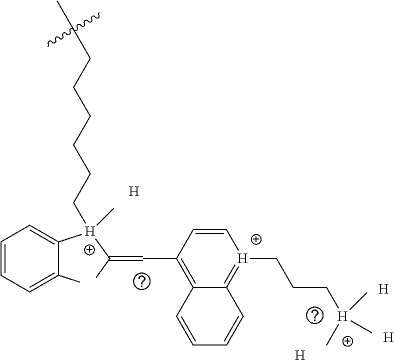 |
| 3 | Proflavine derivative | 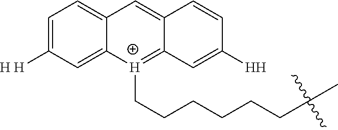 |
| 4 | Acridine orange | 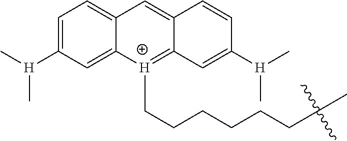 |
| 5 | Ethylene blue derivative | 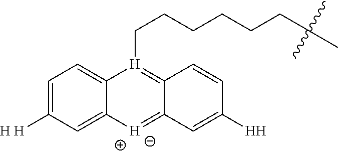 |
| 6 | Ellipticine derivative | 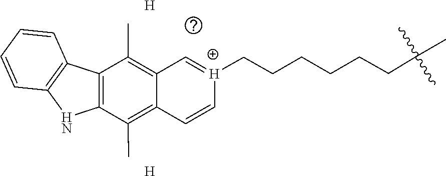 |
| 7 | Phenanthridine derivative | 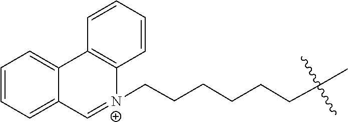 [SMILES: *CCCCCC[n+]1cc2ccccc2c2ccccc21] |
| 8 | Quinolinium derivative | 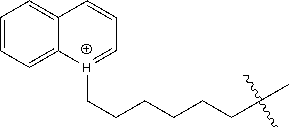 [SMILES: *CCCCCC[H+]1=CC=Cc2ccccc21] |
| 9 | Thalidomide derivative | 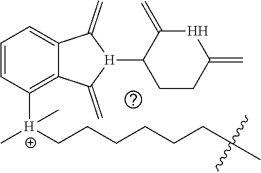 |
| 10 | Naphthalimide derivatives | 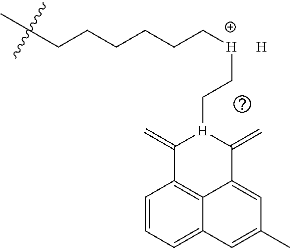 |
| | | R = NO₂, NH₂ |
| 11 | Quinoline derivative | 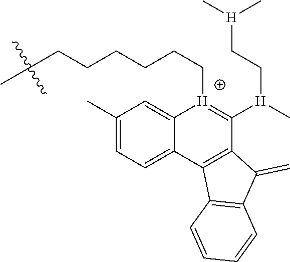 |

Further, the backbone polymer was functionalized with a zwitterionic group to make a zwitterionic polymer. Zwitterionic group has both cationic and anionic groups in the same molecules. These zwitterionic polymers are prepared by functionalizing the backbone polymer with a zwitterionic group or a combination of cationic group and anionic group as described hereinabove.

In a preferred embodiment, taurine and acridine hexyl bromide are used to impart anionic (pTau) and cationic (pAcr) moieties, respectively. Anionic polymer (pTau) is prepared by mixing taurine with a polar solvent, preferably selected from sodium bicarbonate, and then adding this solution to the backbone polymer and stirring at 50 to 80° C. for 12 to 16 hours. The polar solvent is selected from ethanol, methanol, tetrahydrofuran, dioxane, acetonitrile and dimethylsulphoxide

Cationic polymer (pAcr) is prepared by mixing acridine orange base in anhydrous toluene and 1,6-dibromohexane was added to it and refuxed at 120° C. overnight to prepare acridine hexyl bromide. To a solution of the backbone polymer N, N-dimethyl ethylene diamine (87 μl, 0.8 mmol) in THF (10 ml) was added and the mixture was stirred at 50 to 80° C. for 3 to 16 hours. After its dissolution, THF in the reaction mixture was reduced until a viscous solution appeared using rotavapour. Dry dimethylformamide (DMF) was added to this viscous solution, followed by the addition of Acridine (Acr) hexyl bromide. The resulting mixture was stirred at 50 to 80° C. for 12-16 hours and the polymer was triturated thoroughly with deionized water to remove unreacted Acr-Hexyl-Bromide and DMF.

These charged anionic and cationic polymers were engineered into nanofibers by electrospinning to solve the challenges associated with the production of DAMPs. The charged anionic and cationic nanofibrous scaffolds are used individually or in combination. In an alternate embodiment, the same backbone polymer is both positively and negatively charged.

The charged anionic and cationic nanofibrous scaffolds could be prepared in various forms selected from a group consisting of a sheet, bead, bag, insert device, tube, and the like. The size of the nanofibrous scaffolds of a particular form varies depending on blood volume.

In a preferred embodiment, the nanofibrous scaffold is a sheet. Its size depends on blood volume. Preferably, its size is in the range of from 8 to 30 cm².

42 day (D) old pRBCs and their supernatant (supernatent) were treated with pTau and pAcr scaffolds. The treatment helped in reducing the DAMPs significantly within 5 minutes from both pRBCs and supernatant at 4° C. Intermittent treatment on 21^{st} and 28^{th} D of RBCs storage improved human RBCs' quality (higher population of discocytes) at 42 D as compared to untreated, where the 28^{th} D treatment proved better. In mice, the stored RBCs units were treated twice with scaffolds on the 5^{th} and 10^{th}. The findings suggest that cleaning the blood twice maintains the integrity of RBCs and improves their functionality at 14^{th} D. The dual treatment pushed the shelf life of murine RBC by three days (17 Days). The transfusion of 17 D old RBCs recorded a recovery of up to 72 hours with no systemic inflammation. The scaffolds were also tested for hemocompatibility, where they did not interact with blood cells and showed negligible leaching.

These scaffolds (pTau and pAcr) used in the present invention selectively scavenge charged extracellular DNA-histone complex, hemoglobin (Hb), and bioactive lipids. When treated with the charged scaffold, the stored human blood reported a significant reduction in DAMPs at the end of the 42nd D. The scaffold treatment further slowdown the process of ageing in stored pRBCs. The scaffold treatment on the 28th D of storage depicted a lower production of DAMPs and preserved the membrane symmetry of RBC at 42nd D. Interestingly, the mice RBCs that were treated twice with the scaffolds (5th and 10th Day) not only improved the quality of stored blood but enhanced its shelf life by 3 days. The recovery experiments in mice reveal the increased residence time in circulation for treated 17 D old RBCs. Thus, the blood cleaning technology of the present invention that scavenges DAMPs has immense potential in the arena of blood transfusion and can bring significant refinement in the current acceptable blood storage procedure.

Reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized without departing from scope of the embodiments. The invention is described in connection with such embodiments, but the invention is not limited to any embodiment. While only certain features of the invention have been illustrated and described herein, many modifications and changes will occur to those skilled in the art.

### EXAMPLES

### Example 1: Synthesis and Fabrication of Hemocompatible Hydrophobic Charged Polymeric Nanofibers

Materials: PMVEMA-poly(methoxy vinyl ether-alt-maleic anhydride) average Mw-330000 was used in all synthesis procedures. Tetrahydrofuran (THF), tetradecyl amine, taurine, sodium bicarbonate, ethyl acetate, 3,6-bis(dimethylamino) acridine hemi (zinc chloride) salt, benzene, methanol, and ammonium hydroxide, N, N-dimethyl ethylene diamine, 1,6-dibromohexane, sodium sulfate, toluene, dimethylformamide, triton X-100, chamber slides (HI Media), blood collection tubes (BD Vacutainer®), citric acid monohydrate, trisodium citrate dihydrate, sodium dihydrogen phosphate, dextrose, sodium chloride, glucose anhydrous, mannitol, adenine, APTMS (3-(Aminopropyl) trimethoxy silane), glutaraldehyde, cacodylate buffer, osmium tetraoxide, hexamethyldisilane, ethanol, double distilled water and deionized water were used. Unless mentioned otherwise, all chemicals were procured from Sigma-Aldrich.

### Methods:

Synthesis of Anionic (pTau) and Cationic (pAcr) Scaffolds:

Preparation of PMVEMA-C14 (50%): The PMVEMA (500 mg, 3.2 mmol) was added to tetrahydrofuran (THF) (10 ml) in a pressure tube and kept for stirring at 80° C. until dissolution. This was followed by the addition of tetradecylamine (345 mg, 1.6 mmol) prepared in THF (10 ml). The reaction mixture was kept at 80° C. for 3 hours. The solvent was finally removed using rotavapour to get the polymer in dry powder form (800 mg, ˜90% yield).

NMR Data: ¹H-NMR (CD₃OD, 600 MHz), δ:3.0-3.51 (Br, 9H, —OCH₃, —O—CH, PMVEMA and —NH—CH₂ alkyl protons), 1.47 (Br, 2H, alkyl protons), 1.22 (Br, 22H, alkyl protons), 0.845 (t, 3H, alkyl protons). As per NMR data, ˜50% of PMVEMA polymer was substituted with tetradecyl amine. FTIR (cm⁻¹): 3354, 2926, 1858, 1782, 1733, 1697

Preparation of PMVEMA-C14 (50%)-Tau (25%) (pTau): A solution of taurine (Tau) (100 mg, 0.8 mmol) and sodium bicarbonate was prepared (84 mg, 1.0 mmol) in deionized water (1 ml). It was added dropwise to the solution of PMVEMA-C14 (50%) (800 mg) in THF (20 ml) and left for stirring at 80° C. for 16 hours. The reaction mixture was subjected to rotavapour to concentrate. Then, it was sequentially triturated with ethylacetate and deionized water to remove the residual tetradecyl amine and taurine. The final polymer was obtained in a gel form, which was lyophilized to a white powder (720 mg, ˜75% yield).

NMR Data: ¹H-NMR (CD₃OD, 800 MHZ), δ:3.9-4.1 (Br, 1H, taurine), 2.7-3.5 (Br, 5H, —OCH₃, —O—CH, PMVEMA and —NH—CH₂ alkyl protons), 1.95 (Br, 2H, PMVEMA protons), 1.4 (Br, 1H, alkyl protons), 1.1-1.2 (Br, 11.5H, alkyl protons), 0.8 (t, 1.5H, alkyl protons). As per the NMR data, PMVEMA was approximately substituted by 50% of C₁₄H₂₉—NH₂ and ˜25% of taurine. FTIR (cm⁻¹): 3209, 3046, 2946, 1722, 1616, 1037

Preparation of Acridine hexyl bromide: 3,6-bis(dimethylamino) acridine hemi (zinc chloride) salt (10 g, 27.02 mmol) was dissolved in methanol (50 ml), and ammonium hydroxide (50 ml) was added to it. Acridine orange (AO) base was extracted from benzene and dried over sodium sulphate; the AO base (5 g) was recovered from benzene using rotavapour. The AO base (2.65 g, 10 mmol) was dissolved in anhydrous toluene (100 ml), and 1,6-dibromohexane (7.7 ml, 50 mmol) was added to the solution and refluxed overnight. The bright red precipitate in the reaction mixture was filtered and dried (yield-2.1 g, 41.2%).

NMR Data: ¹H-NMR (CDCl₃, 600 MHz) δ:8.7 (s, 1H), 7.9 (d, 2H), 7.08 (m, 2H), 6.75 (s, 2H), 5.0 (t, 2H), 3.4 (t, 2H), 3.3 (s, 12H), 2.048 (m, 2H), 2.029 (m, 2H), 1.96 (m, 2H), 1.94 (m, 2H).

Preparation of PMVEMA-C₁₄ (50%)-Acr (15%): The solution of N, N-dimethyl ethylene diamine (87 μl, 0.8 mmol) in THF (10 ml) was added dropwise to the solution of PMVEMA-C₁₄(50%) (800 mg) in THF (20 ml) in a pressure tube. The mixture was kept for stirring at 80° C. for 16 hours. After its dissolution, THF in the reaction mixture was reduced until a viscous solution appeared using rotavapour. Dry dimethylformamide (DMF) (10 ml) was added to this viscous solution, followed by the addition of Acridine (Acr) hexyl bromide (243 mg, 0.48 mmol) in DMF (5 ml). The resulting mixture was stirred at 50° C. for 16 hours. The polymer was triturated thoroughly with deionized water to remove unreacted Acr-Hexyl-Bromide and DMF. Further, the polymer was lyophilized to an orange powder (800 mg, ˜80%).

NMR Data: ¹H-NMR (CDCl₃, 800 MHz), δ:6.5-8.6 (Br, 0.77H, acridine protons), 3.0-3.5 (Br, 4H, PMVEMA protons), 0.98-1.7 (Br, 9.5H, alkyl protons), 0.87 (Br, 3H, alkyl protons). As per the NMR data, approximately 50% of C₁₄H₂₉—NH₂ and ˜11% of acridine hexyl bromide was substituted on PMVEMA polymer.

Electrospinning conditions: The electrospinning solutions were prepared by dissolving 20% (w/v) pTau and 15% (w/v) pAcr in dry DMF at room temperature (RT), separately. The two solutions were stirred overnight before electrospinning. During the electrospun procedure, a 2-mL plastic syringe (Dispo Van, India) and a blunt 22 G metallic needle (Dispo Van, India) were used. The solutions were pumped via a syringe pump at a constant flow rate of 0.5 ml/h (pTau) and 0.3 ml/h (pAcr), separately. The two electrospun nanofibre scaffolds were collected on an aluminium foil placed 15 cm away from the needle (where 15 kV voltage was applied). All experiments were carried out at 25° C. and less than 55% relative humidity (RH). The electrospun mats with a length and width of approximately 10×10 cm were stored in a desiccator until further use.

Field Emission Scanning Electron Microscopy (FESEM): The morphology of the electrospun scaffolds (pTau and pAcr) were investigated by FESEM (Carl Zeiss MERLIN VP compact). Dried electrospun mats were mounted on aluminium stubs and sputter-coated with gold (Pelco® SC-7 Auto sputter coater). The gold coated mats were imaged at a voltage of 2 kV. The scaffolds treated with pRBCs were imaged to investigate the adherence of blood cells.

Contact angle measurement: The wettability of electrospun scaffold was studied using the sessile drop method. The scaffolds were cut uniformly and placed on a glass slide before the measurement. Water droplets (5 μL) ˜2 mm in diameter were dropped on the electrospun membrane, and the images were acquired. The images were processed using ImageJ software (V1.53).

Hemolysis Assay: A hemolysis assay was performed to evaluate the haemolytic nature of cationic and anionic (pTau-pAcr) scaffolds. The blood from a human volunteer was collected in a tube containing anticoagulant sodium citrate. The collected blood was centrifuged (500 G, 10 min), and the supernatant was discarded. These washed RBCs were used to prepare 2% hematocrit in saline and added to scaffold fixed in chamber slides (8 cm²) prewashed with double distilled water. The RBCs were further resuspended in saline and pelleted down. This setup was kept on an incubator shaker (37° C. and 100 rpm) for 1 hour. The hematocrit after 1 hour was transferred to a 2 ml centrifuge tube and centrifuged at 500 G for 10 minutes at room temperature. 100 μl of supernatant was collected and transferred to a 96-well plate. Thus, this procedure involves hemoglobin quantification in the supernatant due to lysed RBCs treated with scaffolds for 1 hour. 0.1% Triton-X100 in 2% haematocrit (same volume) was taken as the positive control, while pure 2% haematocrit served as the negative control. A 100% lysis sample of the untreated RBC specimen was prepared with 0.1% Triton-X100 in 2% hematocrit. The concentration of Hb in a 100% lysed RBCs sample was determined by cyanmethemoglobin (Drabkin's) method, which is considered as 100%. Afterward, a series of dilutions was prepared from lysed sample to generate a standard curve. The level of hemolysis of the pTau-pAcr-treated RBCs is then derived from the standard curve using Drabkin's method. For Drabkin's method, the absorbance was recorded at 540 nm using Varioskan LUX Multi-Mode Microplate Reader (Thermos fisher, Massachusetts, USA). The hemolysis (%) was calculated as:

$$\frac{Average OD of Blood Supernatant}{Average OD of 100 \% Hemolysis control} \times 100$$

Polymer Leaching test: pAcr scaffolds (8 cm²) were fixed on the chamber slides and incubated with double distilled (DD) water. The scaffolds were incubated for 30 minutes, followed by a short incubation of 5 minutes. The DD was removed after both incubations and lyophilized to recover the leached pAcr from the scaffolds. The lyophilized pAcr was dissolved in DMF and estimated using a fluoro spectrophotometer (Horiba Fluro log QM, France). Fluorescence intensities were recorded as a function of time with excitation at 502 nm and emission at 535 nm. The Leaching (%) was determined via a pure pAcr polymer standard curve prepared at the excitation and emission wavelengths mentioned above.

Discussion: High molecular weight (Mw) PMVEMA (average Mw-3,30,000) was selected to functionalize anionic and cationic counterparts to scavenge soluble DAMPs produced in the pRBCs. As the polymers were expected to scavenge the DAMPs from aqueous media, the polymers must be hydrophobic in nature. The hydrophobicity was imparted by conjugating tetradecylamine to PMVEMA via amide linkage. The tetradecyl amine conjugation was followed by the functionalization of the polymer with taurine and acridine derivative resulting in charged hydrophobic polymers, i.e., pTau and pAcr. Sulphonic acid in taurine imparts anionic charge to pTau while Acridine provides cationic charge to pAcr. The anionic group offers a higher affinity against cationic species like amines, guanidine. On the flip side, Acridine acts as an efficient intercalator and scavenges nucleic acids selectively without interacting with the cell membrane. This functionalization was carried out via one-pot synthesis. 50% of the anhydride groups were conjugated with tetradecyl amine (Poly-C₁₄-50%) followed by functionalization of taurine (anionic) and acridine hexyl bromide (cationic). Surface charge measurements confirmed the overall negative and positive charge of pTau and pAcr, respectively (FIG. 1c).

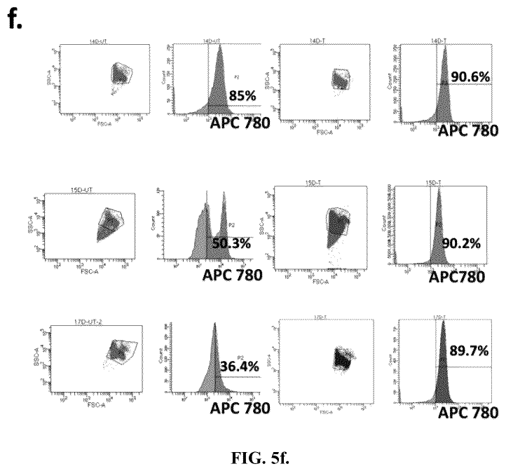

FTIR peaks at 2926, 1782, 1732 cm-1 confirmed the presence of alkyl chain, maleic anhydride, and amide bonds. The NMR findings substantiate ˜50 and 25% substitution of tetradecyl amine and taurine in pTau while pAcr constituted ˜50% of tetradecyl amine and ˜11% of Acr-hexyl bromide substitution over PMVEMA (FIG. 7). In order to develop a biomaterial platform, pTau and pAcr were then subjected to electrospinning to engineer nanofibre at an optimized concentration of 20% and 15% (w/v). The polymer flow rate and the voltage were maintained at 0.3 ml/hour and 15-20 kV, while the needle to collector distance was fixed at 15 cm. The electrospun scaffolds were imaged under FESEM (FIG. 1b), confirming that the fibers were intact, stable, and bead-free.

Since the electrospun charged nanofibers were expected to be hydrophobic in nature, their extent of wettability was evaluated by contact angle measurements. The water contact angles for pAcr (140.033±4.41°) and pTau (144.33±5.507°) were recorded (FIG. 1b). The high contact angles (super hydrophobic domain) further confirm the incorporation of the aliphatic chain in polymers that induced hydrophobicity. Imparting hydrophobicity to the polymers was paramount as it helped the nanofibres to retain their structural integrity in aqueous media (pRBC unit).

To explore biocompatible features of engineered polymers, it was critical to substantiate that none of the polymer components leached into the blood during treatment. To demonstrate this, 8 cm² nanofibre pAcr was weighed (6.75 mg) and incubated with 1 ml of distilled water for 30 minutes at ambient temperature. The incubated water was collected after 30 minutes and lyophilized. The lyophilized product was processed via fluorescence spectroscopy (FIG. 1), and 0.015 mg (0.2%) of pAcr was quantified. pAcr was further subjected to sequential incubation with distilled water for 5 min; the findings confirmed 0.03% pAcr in water. Similarly, pTau was also examined and tested via NMR and FTIR spectroscopy, but nothing significant was detected (data not shown) to confirm leaching. The leaching data testified high stability of nanofibers in aqueous media. To further exclude any interference of leached polymer with the pRBC units, the scaffolds were pre-treated with autoclaved distilled water for 30 minutes.

The biocompatibility perspective was further investigated by incubating 24 cm² scaffold pTau and pAcr with 42 D human pRBCs and its supernantent for 30 mins. After collecting RBCs and supernatent, the dried scaffolds were imaged under FESEM. The FESEM images showed that the blood cells did not adhere to the scaffold (FIG. 1d). This experiment further validated that scaffold does not interfere with RBCs membrane integrity/health and is highly hemocompatible to pursue scavenging studies. The hemolysis assay also cemented the viability of the scaffolds. The nanofiber scaffolds were incubated with human RBC for 1 hour at 37° C. (FIG. 1e), and the hemolysis was estimated. The statistics revealed ≤1% hemolysis for pTau while it was ≤3% in the case of pAcr. These findings assured scaffold's compatibility with RBC, which eventually added to its scavenging potential.

### Example 1A: Charged Nanofibrous Sheets for Scavenging DAMPs from Both Old Stored RBCs Units and their Supernatant

Materials: Materials Used were Same as Those in Example 1
Methods: Synthesis of Anionic (pTau) and Cationic (pAcr) Scaffolds: These were Prepared in the Same Manner as in Example 1

Fabrication of electrospun nanofibrous sheets: The electrospinning solutions were prepared by dissolving 20% (w/v) pTau and 15% (w/v) pAcr in dry DMF at room temperature (RT), separately. The two solutions were stirred overnight before electrospinning. During the electrospun procedure, a 2-mL plastic syringe (Dispo Van, India) and a blunt 22 G metallic needle (Dispo Van, India) were used. The solutions were pumped via a syringe pump at a constant flow rate of 0.5 ml/h (pTau) and 0.3 ml/h (pAcr), separately. The two electrospun nanofibrous sheets (Tau-NFS and Acr-NFS) were collected on an aluminium foil placed 15 cm away from the needle (where 15 kV voltage was applied). All experiments were carried out at 25° C. and less than 55% relative humidity (RH). The electrospun mats with a length and width of approximately 10×10 cm were stored in a desiccator until further use.

Field Emission Scanning Electron Microscopy (FESEM): The morphology of the Tau-NFS and Acr-NFS were investigated as set out in Example 1.

Contact angle measurement: The wettability of Tau-NFS and Acr-NFS was studied using the sessile drop method. The scaffolds were cut uniformly and placed on a glass slide before the measurement. Water droplets (5 μL) ˜2 mm in diameter were dropped on the electrospun NFS, and the images were acquired. The images were processed using Image software (V1.53).

Hemolysis Assay: A hemolysis assay was performed to evaluate the haemolytic nature of Tau-AcrNFS as set out in Example 1. The blood from a human volunteer was collected in a tube containing anticoagulant sodium citrate and centrifuged (500 G, 10 min), and the supernatant was discarded. These washed RBCs were used to prepare 2% hematocrit in saline and added to Tau-AcrNFS fixed in chamber slides (16 cm³) prewashed with double distilled water. The RBCs were further resuspended in saline and pelleted down. This setup was kept on an incubator shaker (37° C. and 100 rpm) for 1 hour. The hematocrit after 1 hour was transferred to a 2 ml centrifuge tube and centrifuged at 500 G for 10 minutes at room temperature. 100 μl of supernatant was collected and transferred to a 96-well plate. Thus, this procedure involves hemoglobin quantification in the supernatant due to lysed RBCs treated with scaffolds for 1 hour. 0.1% Triton-X100 in 2% haematocrit (same volume) was taken as the positive control, while pure 2% haematocrit served as the negative control. A 100% lysis sample of the untreated RBC specimen was prepared with 0.1% Triton-X100 in 2% hematocrit. The concentration of Hb in a 100% lysed RBCs sample was determined by cyanmethemoglobin (Drabkin's) method, which is considered as 100%. Afterward, a series of dilutions was prepared from lysed sample to generate a standard curve. The level of hemolysis of the Tau-AcrNFS-treated RBCs is then derived from the standard curve using Drabkin's method. For Drabkin's method, the absorbance was recorded at 540 nm using Varioskan LUX Multi-Mode Microplate Reader (Thermos fisher, Massachusetts, USA).

Polymer Leaching test: Acr-NFS (8 cm²) was fixed on the chamber slides and incubated with optisol (AS-5) solution [composition: sodium chloride (8.77 mg/mL), glucose anhydrous (8.18 mg/mL), mannitol (0.01% w/v) and adenine (0.03 mg/mL)]. The sheets were incubated for 2 hours. The optisol solution was removed after both incubations and lyophilized to recover the leached poly-Acridine from the sheets. The lyophilized poly-Acridine was dissolved in DMF and estimated using a fluoro spectrophotometer (Horiba Fluro log QM, France). Fluorescence intensities were recorded as a function of time with excitation at 502 nm and emission at 535 nm. The Leaching (%) was determined via a pure poly-Acridine polymer standard curve prepared at the excitation and emission wavelengths mentioned above.

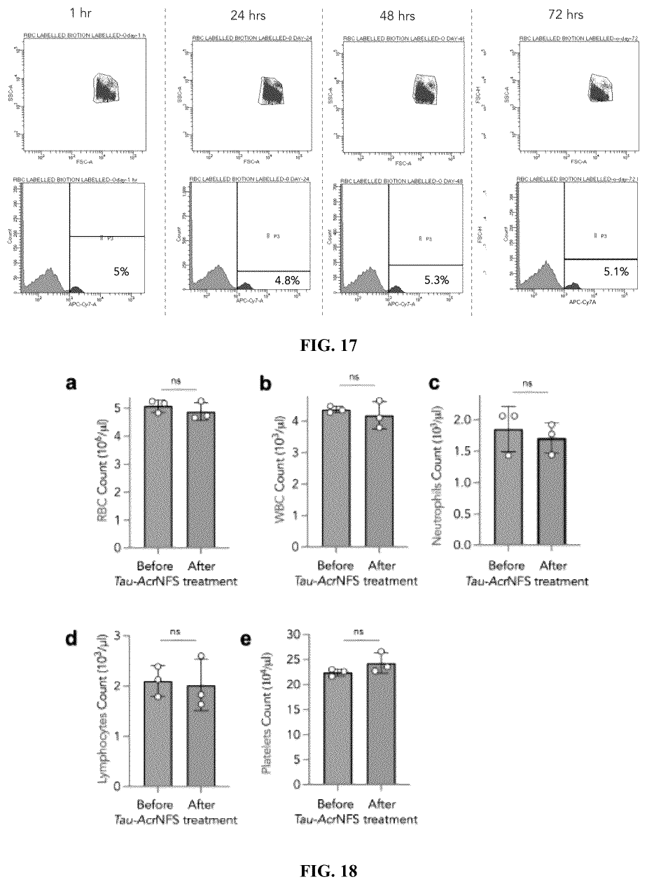

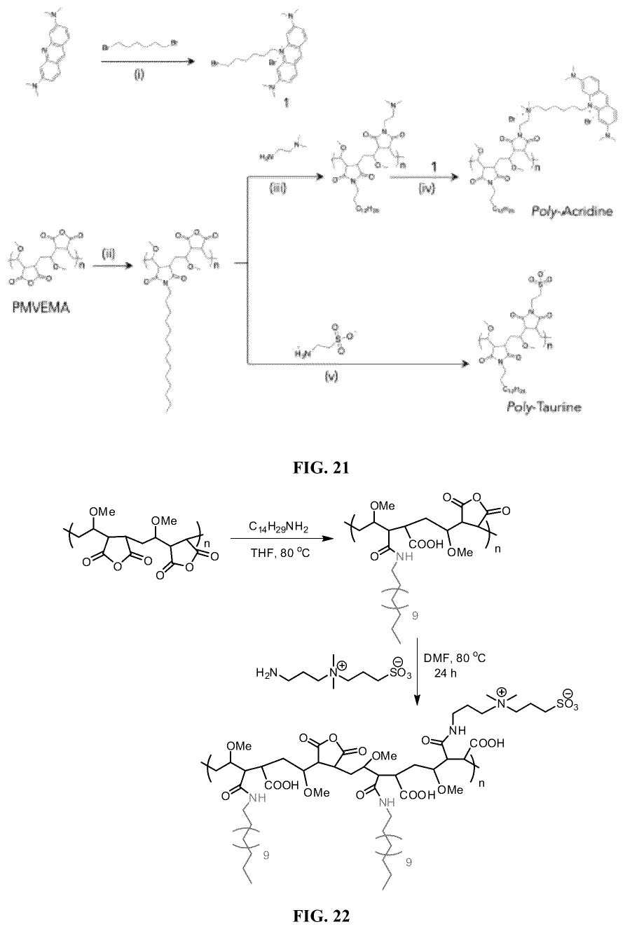

Discussion: Charged electrospun nanofibrous sheets efficiently scavenge DAMPs: To synthesize polymers with anionic/cationic charges to generate electrospun nanofibrous sheets, poly(methyl vinyl ether-alt-maleic acid) [PMVEMA, average Mw: 330,000] was selected, to which 50% of tetradecylamine was functionalized to impart hydrophobic nature to the polymer. Subsequently, either 25% of taurine or 11% of hexyl acridine were functionalized to obtain anionic (poly-Tau) and cationic (poly-Acr) polymers, respectively. In addition to furnishing cationic charge, acridine can also bind DNA by intercalation, which enhances the ability of Acr-NFS to scavenge extracellular DNA. The detailed synthesis scheme is provided in FIG. 21. Anionic (Tau-NFS) and cationic (Acr-NFS) electrospun nanofibrous sheets were generated through conventional electrospinning of poly-Taurine and poly-Acridine, respectively (Methods). Tau-NFS and Acr-NFS are comprised of nanofibers with 100-200 nm width and >50-100 micron length (FIG. 1Ab). Surface charge measurements confirmed the overall negative and positive charge of Tau-NFS and Acr-NFS, respectively (FIG. 1Ac). Furthermore, contact angle measurements show that both NFS are hydrophobic (FIG. 1Ad), which is critical to prevent aqueous fluid absorption. A 2-h incubation of Tau-NFS and Acr-NFS in optisol (AS-5) solution that is used as a preservative for storing RBCs suggested that NFS are stable in preservative solution, and the quantification of leached polymers shows that neither polymers have any significant leaching (<0.2%). Scanning electron microscope images of Tau-NFS and Acr-NFS post-incubation with stored RBCs show that RBCs or neutrophils do not adhere to NFS (FIG. 1Ae); hence, there will not be any loss of RBC count due to the NFS treatment. Furthermore, to understand whether treatment of Tau-NFS and Acr-NFS can cause cell loss, we performed a complete blood count (CBC) before and after treatment with Tau-NFS and Acr-NFS. Quantifying the number of RBCs, white blood cells, platelets, neutrophils and lymphocytes before and after treating with NFS (FIG. 18) revealed that there is no loss of cells suggesting that these nanofibrous sheets do not cause any cell death or cell capture. Additionally, the hemolysis rate was measured upon incubation of RBCs with Tau-AcrNFS at 37° C. for 1 h. A<1% of hemolysis was observed for Tau-Acr-NFS (FIG. 1Af). Cumulatively, these results suggest that Tau-AcrNFS are stable and do not cause significant hemolysis.

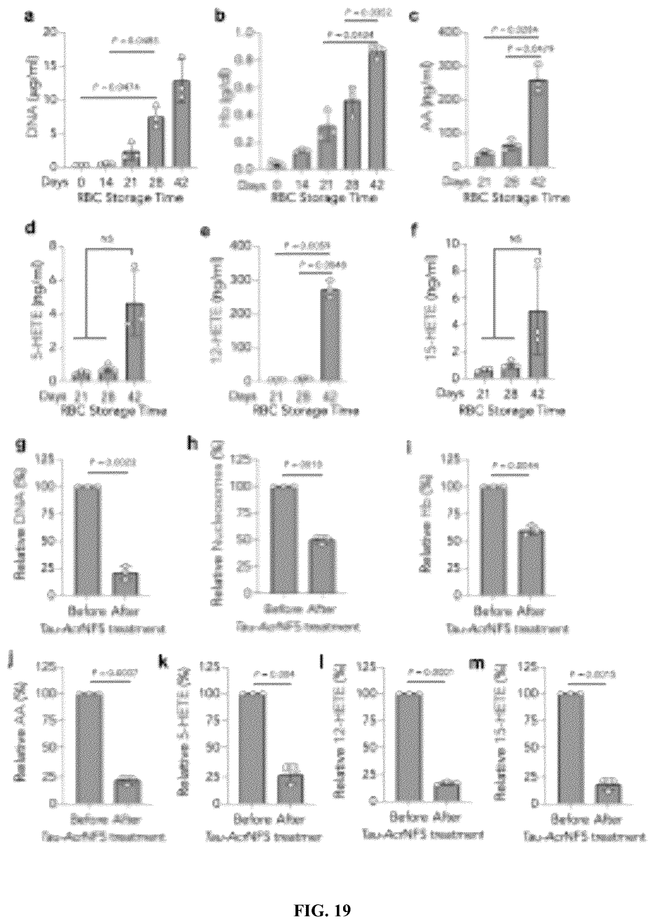

Before testing the DAMPs-scavenging efficacy of NFS, the production of DAMPs (extracellular DNA, Hb, and PUFAs) in stored human non-leukoreduced RBCs in a time-dependent manner for up to 42 days was quantified. Various analytical techniques such as PicoGreen assay, ELISA, Drabkin's assay, and LC-MS were used to quantify extracellular DNA, nucleosomes, Hb, and PUFAs, respectively (Methods). Quantitative elucidation of the storage lesion revealed that DAMPs, including extracellular DNA (FIG. 19a), Hb (FIG. 19b), and PUFAs such as arachidonic acid (AA, FIG. 19c) and hydroxyeicosatetraenoic acids (5-HETE, 12-HETE, and 15-HETE, FIG. 19d-f) increase over time. For example, on the 42nd day, extracellular DNA and Hb concentration increased by ˜150-fold and 40-fold respectively, compared to the 0th day.

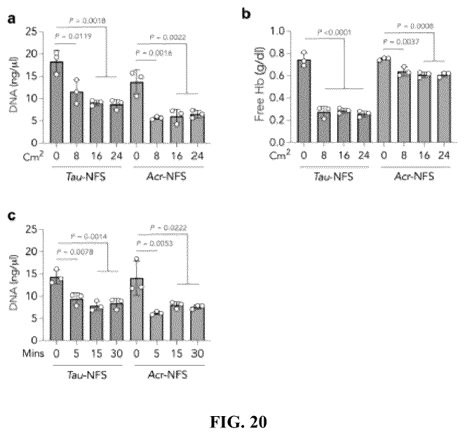

At the onset, we have systematically optimized the required surface area of nanofibrous sheets and incubation time to scavenge DAMPs. One milliliter of RBCs (42 days stored) were added to the different surface areas of sheets (8, 16, and 24 cm²) and incubated for 30 min at 4° C. Quantification of extracellular DNA suggested that both nanofibrous sheets could scavenge DNA efficiently with an 8 cm2 sheet, and above 8 cm2 up to 24 cm2 did not increase the scavenging capacity (FIG. 20a). Subsequently, RBCs were incubated on 8 cm2 nanofibrous sheets for 5, 15, and 30 min to identify the optimum incubation time. Data suggests that Tau-NFS and Acr-NFS could scavenge the DNA within 5 min of incubation, and more prolonged incubation did not enhance scavenging capacity (FIG. 20b).

To scavenge all DAMPs (extracellular DNA, nucleosomes, Hb, and PUFAs) from old RBCs, 1 ml of 42 days-stored RBCs were incubated with cationic/anionic nanofibrous sheets (Tau-AcrNFS) for 5 min at 4° C. Data in FIG. 19g-m suggests that Tau-AcrNFS are efficient in scavenging DAMPs. Upon incubation, nanofibrous sheets scavenged 80% of DNA (FIG. 19g), 50% of nucleosomes (FIG. 19h), 45% of Hb (FIG. 19i), and 75-80% of PUFAs including AA, 5-HETE, 12-HETE, and 15-HETE (FIG. 19j-m). These results suggest that Tau-AcrNFS can efficiently scavenge and significantly reduce the concentrations of DAMPs in 42 days of stored old RBCs. SEM images of Tau-NFS and Acr-NFS posttreatment with stored RBCs (FIG. 1Ae) shows a gummy layer on the surface, which could be due to the accumulation of sequestered proteins and lipids on the nanofibrous sheets.

### Example 2: Charged Nanofibre Scaffolds Scavenge DAMPs from Both Old Stored RBCs Units and their Supernatant

### Methods:

Packing of Human RBC (pRBC) as Non-leukoreduced (NLR) and Leukoreduced (LR) units: NLR and LR RBC units were prepared from human whole blood (both male and female, n=3) of different blood groups stored in 20 ml vacutainers containing citrate-phosphate-dextrose with adenine (CPD-A) (contains citric acid monohydrate-3.577 mg/mL, trisodium citrate dihydrate-29.972 mg/mL, sodium dihydrogen phosphate-2.496 mg/mL and dextrose-25.5 mg/mL). Whole blood was centrifuged for 20 minutes at 500 G, and the supernatant was removed. The removal of supernatant was followed by the addition of optisol (AS-5, preservative) (contains Sodium chloride—8.77 mg/mL, Glucose anhydrous—8.18 mg/mL, Mannitol—0.01% w/v and Adenine—0.03 mg/mL) to obtain a hematocrit of 65% (approx.), termed as NLR blood.

Similarly, LR RBCs were processed from whole blood with an additional step of leukoreduction within 24 hours of blood collection via leuko-filters (Acrodisc® PSF WBC filters, Merck). After leukoreduction, AS-5 preservative was added to get 65% hematocrit termed as LR blood. The LR and NLR blood were then transferred to 2 ml tubes for packing, leaving a small space for residual air, and stored at 4° C. (dark) until further use.

In vitro efficacy of pTau and pAcr to scavenge DAMPs in human RBCs units: pRBCs (Both NLR and LR) stored for 42 D and their supernatants termed as 42 D-RBCs and 42 D-T respectively were treated with pTau and pAcrscaffolds individually as well as in combination to scavenge DAMPs up to 42 D i.e. the shelf life of RBCs. Also, for the intermediate cleaning, the NLR and LR blood were treated on the 21^{st} and 28^{th} Day of storage with pTau, pAcr individually and in combination. In both the treatment condition, the nanofibre scaffolds were pre-wetted with distilled water for 30 minutes before the addition of RBCs. The total surface area of the scaffold was kept 24 cm² for each experiment. RBCs with scaffolds were treated at 4° C. for 5 min to keep the cold chain storage. After the scavenging procedure, the RBCs were again transferred in autoclaved 2 ml Eppendorf tubes, leaving a small residual space, and stored till the 42^{nd} Day, and its supernatant was collected for further analysis, including biochemical assays. The untreated group was not given any treatment during or after the storage period, and the supernatant was collected at 42 D to compare with the treated group.

Quantification of Free Hb scavenging experiment using Cyanomethaemoglobin (Drabkin's) method: Drabkin's reagent lyses red blood cells and oxidizes all forms of Hb, except for the minimally present sulfhaemoglobin, to the stable HiCN (Didar, T. F. et al. Improved treatment of systemic blood infections using antibiotics with extracorporeal opsonin hemoadsorption. Biomaterials 67, 382-392 (2015)). The supernatant of the stored RBCs unit (NLR and LR, scaffold-treated or untreated) was diluted (1:10) with Drabkin's reagent (Sigma, St. Louis, MO, USA). Human Hb diluted with Drabkin's reagent (1:10) (Sigma Aldrich, USA) was used to prepare the standards (0-40 mg/dl) and the calibration curve. The samples (LR and NLR) and standards were incubated in the dark at room temperature for 15 minutes. Absorbance was recorded at 550 nm using a Multi-Mode Microplate Reader (Thermo fisher, Massachusetts, USA). The Hb concentration of each sample (LR and NLR) was calculated from the human Hb calibration curve.

Quantification of free DNA by Pico green Assay: RBC supernatants (NLR and LR) from scaffold-treated and untreated groups were thawed on ice. Supernatants were diluted (1:50) with PicoGreen® reagent and Tris-EDTA (TE) buffer (Quant-iT™ PicoGreen™ dsDNA Assay Kit, Invitrogen, USA), followed by 5 minutes of incubation in the dark. Free DNA estimation was performed using a fluoro spectrophotometer (Horiba Fluro log QM, France). PicoGreen® circumvents contributions from interfering substances and exhibits an emission maximum at 530 nm when explicitly bound to dsDNA (unbound PicoGreen® reagent exhibits minimal fluorescence in solution) (Land, W. G. Transfusion-related acute lung injury: The work of DAMPs. Transfus. Med. Hemotherapy 40, 3-13 (2013)). DNA was quantified via a standard curve ranging from 0-10 ng/μL.

Quantification of nucleosomes: Stored RBCs supernatants were diluted in PBS (1:5). The Anti-Histone-Biotin-Monoclonal antibody (cloneH11-4) and Anti-DNA-POD-Monoclonal antibody from mouse (cloneMCA-33) was used for quantifying nucleosomes by ELISA (Catalog No. 11920685001, Cell Death Detection kit, Roche, Indianapolis, IN), according to manufacturer's instructions

### Bioactive Lipid Estimation by LCMS:

Chemicals and Standards: Eicosanoids; 5(S)-HETE, 12(S)-HETE, 15(S)-HETE, and Arachidonic acid (AA) were purchased from Cayman Chemical Co. (Ann Arbor, Michigan, USA). 5(S)-HETE-d8 (Cayman Chemical Co) was used as an internal standard (IS) for this study. Acetonitrile (Baker Analyzed® LC-MS reagent), water (Baker Analyzed® LC-MS reagent), formic acid, ethanol, ethyl acetate (EtOAc), and glacial acetic acid were used in sample preparations for LC-MS/MS measurements.

Preparation of Stock Solution: 5-(S)-HETE, 12(S)-HETE, 15(S)-HETE, AA, and internal standard 5(S)-HETE-d8 were constituted in ethanol by the provider. 5, 12 and 15 (S)-HETE were further processed and serially diluted to obtain solutions ranging from 0.05 ng/ml to 100 ng/ml. AA solutions ranged from 0.5 ng/ml to 1000 ng/ml. The working concentration of the pure IS was 1000 ng/ml.

Sample Preparation: Lipid extraction from stored RBCs supernatant was performed by gently mixing the samples with 0.5 mL of EtOAc containing 0.13% acetic acid (v/v). All samples were spiked with 5 μl of the internal standard (IS). After 10 min of vigorous shaking (1500 rpm), samples were centrifuged (10,000 rpm, 10 min, 4° C.), and 450 μl of the organic layer was transferred to fresh tubes and evaporated in speed vac (Scan Vac, Labogene, Demark) at 4° C. The dry residue was reconstituted in 25 μL of nitrogen purged EtOH and then directly injected (2 μl) into the LC-MS/MS system.

LC-MS/MS conditions: HPLC analysis was done using a Shimadzu Prominence HPLC system. The instrument was equipped with a communication module (CBM-20A), degassing unit (DGU-20A5R), solvent delivery units (LC-30AD), column oven (CTO-20AC) and an auto sampler (SIL-30AC). The temperature of the column oven was maintained at 40° C. while the autosampler was set at 4° C. The lipids were separated using a Acquity UPLC® BEH C18 1.7 μm column (2.1×50 mm). The mobile phase comprised of solvent A, i.e., water with 0.1% formic acid (FA) and acetonitrile with 0.1% FA as solvent B. The flow rate was maintained at 0.2 ml/minutes and the injection volume was 2 μl. The gradient for lipid elution (20.10 min) was set as: 50% A and 50% B for one minute. This was followed by a linear gradient to 20% A and 80% B for 8 min to elute 5, 12 and 15 HETE. This was further followed by a linear gradient to 100% B for 2 minutes and kept for 5 minutes to elute AA. Finally, the column was equilibrated at 50% A and 50% B for 4 minutes before next injection. LC-MS analysis was performed QTRAP 5500 (Sciex, Framingham, Massachusetts, USA), triple quadrupole mass spectrometer. The operating parameters for mass spectrophotometer were as follows: declustering potential (DP): −209.8 V, entrance potential (EP): −5.7 V, collision cell exit potential (CXP): −16.9 V, ion spray voltage: −4500 V, source temperature: 500° C., curtain gas: 35 psi, ion source gas 1:40 psi and ion source gas 2:50 psi. All the spectra of lipids were recorded and quantified in multiple reaction monitoring (MRM) mode. Negative electrospray ionization was employed for all the lipids and the deuterated internal standard. The ion Q1/Q3 transitions and other parameters can be seen in table below:

TABLE 1
Quantification of bioactive lipids in blood

| | | | Ion | DP | EP | CE | CXP |
|---|---|---|---|---|---|---|---|
| Species | Q1 (m/z) | Q3 (m/z) | Mode | (Volts) | (Volts) | (Volts) | (Volts) |
| Arachidonic | 302.900 | 259.300 | Negative | −209.800 | −5.700 | −16.200 | −16.900 |
| Acid (AA) | | | | | | | |
| 5-HETE | 319.00 | 115.00 | Negative | −127.00 | −3.900 | −18.00 | −7.200 |
| 15-HETE | 319.00 | 219.00 | Negative | −158.400 | −5.100 | −17.200 | −11.900 |
| 12-HETE | 319.00 | 179.200 | Negative | −135.500 | −8.100 | −19.00 | −10.900 |
| 5-HETE-d₈ | 327.200 | 116.00 | Negative | −127.00 | −3.900 | −18.00 | −7.200 |

Discussion: The US regulation states that pRBCs can be stored up to 42 Days (D) for transfusion. Post 42 D, the increased storage lesions (DAMPs) pose a fatal risk to the patient. The storage lesions (extracellular DNA, nucleosomes, free Hb, bioactive lipids) can collectively or individually induce immunomodulatory response.

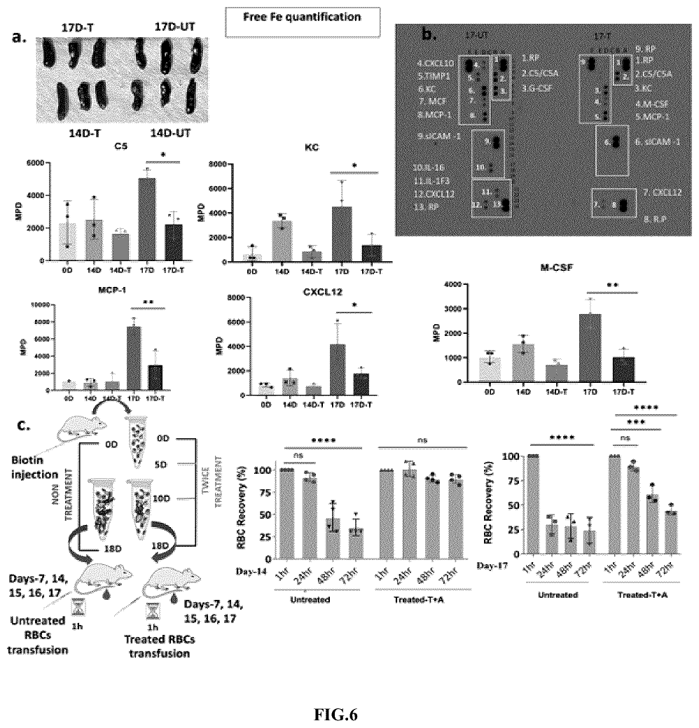

To commence with, the extracellular DNA, free hemoglobin, and bioactive lipids were measured from the supernatant of stored human RBCs (NLR-Non-Leukoreduced, LR-Leuko reduced units) for up to 42 D. To begin with, the extracellular DNA is usually present in the form of neutrophilic extracellular traps (NETs). NETs are loosened complex of methylated histones and DNA. The NLR units recorded a significant increase in extracellular DNA from 21 D to 28 D (from ˜2.31 to ˜7.3 ng/μl) and28 D to 42 D (from ˜7.3 ng/μl to 13.3 ng/μl) (FIG. 8A). The overall spike of ˜150 folds was observed from 0 D to 42 D in NLR blood, which completely complimented the other reports (Cancelas, J. A. et al. Additive solution-7 reduces the red blood cell cold storage lesion. Transfusion 55, 491-498 (2015)).

On the contrary, Leukoreduced RBCs (LR units) showed a gradual increase in the production of extracellular DNA from 0 D to 42 D (˜0.05 ng/μl to ˜0.3 ng/μl) but compared to NLR; it was less by 30-folds (FIGS. 8A and 8B). This was expected as neutrophils are the predominant source of extracellular DNA, that were initially removed using leukofilters (Rapido, F. et al. Prolonged red cell storage before transfusion increases extravascular hemolysis. J. Clin. Invest. 127, 375-382 (2017) (FIGS. 8A and 8B). A similar trend has been witnessed in free hemoglobin (Hb) production in NLR stored units. The free Hb increased up to 40 folds (˜0.03 g/dl to ˜0.8 g/dl) from 0 D to 42 D (FIG. 8A). Similarly, in the LR units, there was limited hemolysis (˜0.03 g/dl to˜0.45 g/dl), but it was not as significant as in NLR units (FIGS. 8A and 8B). This also gives an idea that removing leucocytes (via leucoreduction filters) reduced the formation of DAMPs associated with them, but it ultimately had no control over the accumulation of RBC-associated DAMPs.

Parallelly, the accumulation of lipids like Arachidonic acid (AA), 5-HETE,12-HETE, and 15-HETE in the supernatant (LR and NLR) has been identified and reported previously (Yoshida, T., Prudent, M. and D'Alessandro, A. Red blood cell storage lesion: Causes and potential clinical consequences. Blood Transfus. 17, 27-52 (2019)). It is recorded that all these lipids reach a relative maximum concentration on Day 42 of storage (Fuchs, T. A. et al. Neutrophils release extracellular DNA traps during storage of red blood cell units. Transfusion 53, 3210-3216 (2013)). In our studies, all four non-polar lipids were quantified through LCMS that showed a significantly elevated level from 28 D to 42 D of RBCs storage (LR and NLR). AA, one of the most common omega-6 PUFAs involved in cellular signalling and inflammation, increased exponentially in NLR units during storage from 28 D (˜66 ng/ml) to 42 D (˜258 ng/ml) (FIG. 8A). The rapid production of AA is also contributed by the high activity of phospholipase A2 that releases AA present in the sn-2 position of membrane phospholipids (Vlaar, A. P. J. et al. Accumulation of bioactive lipids during storage of blood products is not cell but plasma derived and temperature dependent. Transfusion 51, 2358-2366 (2011)). In LR units, the increase in production on 42 D (˜111 ng/ml) was approximately two folds higher than 28 D (˜63 ng/ml), affirming the formation of lesions associated with RBCs (FIG. 8B). The oxidation products of AA are known as the eicosanoids, a class of bioactive lipids mainly produced through enzymatic activity (Silliman, C. C. et al. Identification of lipids that accumulate during the routine storage of prestorage leukoreduced red blood cells and cause acute lung injury. Transfusion 51, 2549-2554 (2011)). Cyclooxygenases convert AA into prostaglandins, whereas lipoxygenase (LOX) activity leads to the production of HETEs and leukotrienes. Cytochrome P450s (CYPs) is also reported to aid the production of HETEs (D'Alessandro, A. and Zolla, L. Biochemistry of red cell aging in vivo and storage lesions. Haematologica 7, 389-396 (2013)). Our study found that HETEs significantly increased with the duration of human RBC storage and coincided with the AA generation. 12-HETE (NLR units) recorded a significant increase in its concentration from 28^{th} to the 42^{nd} Day of storage (˜7 ng/ml to˜269 ng/ml). However, this increase was not observed in LR (FIG. 8B). The probable reason for this observation can likely be attributed to the 12-LOX activity of WBCs in NLR units. The resembling spike in concentration was not consistent for 5- and 15-HETE in stored human RBC units, and the trend was similar for both LR and NLR units (FIGS. 8A and 8B). The kinetics data gave comprehensive insight about the DAMPs production in stored RBCs units with respect to time.

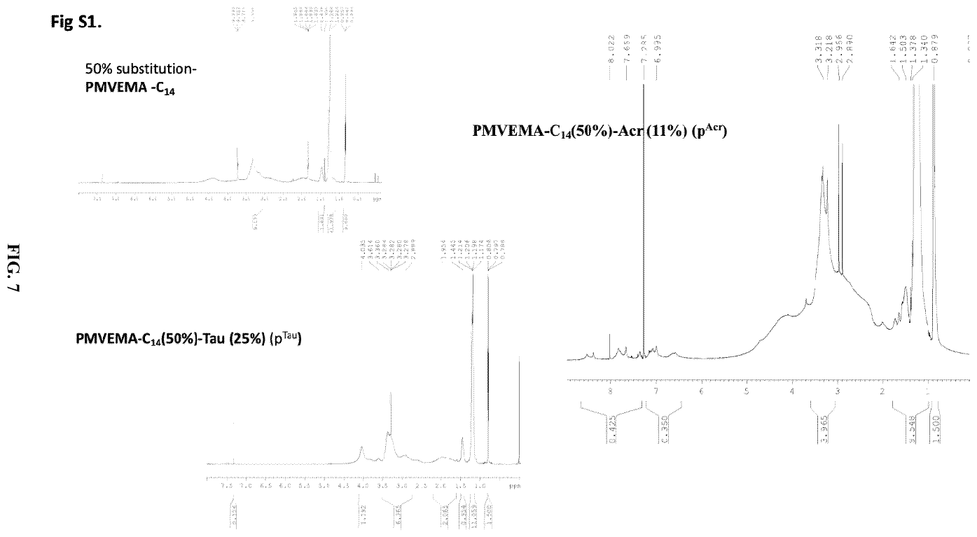

The stored 42 D RBCs supernatant (supt) is a cocktail of charged species ranging from negatively charged extracellular DNA, bioactive lipids including arachidonic acid and eicosanoids to positively charged free hemoglobin, histones. The dominance of charged species was the principal factor that led to the design of a charged biomaterial that could scavenge them. pAcr scaffold was exclusively designed to bind the DNA via intercalation, while pTau hunts positively charged histones. Extracellular DNA in 42 D NLR stored blood supt was selected as a marker to optimize the scaffold surface area (8 cm² to 24 cm²) and incubation time (5 minutes-30 minutes) to scavenge the maximum DAMPs at 4° C. (without breaking the cold chain). The data suggested that 8 cm² of pAcr could decrease extracellular DNA from 13 ng/μl to 5 ng/μl within 5 min from 1 ml of the supernatant (FIG. 9). pTau scaffolds also reported a similar trend.

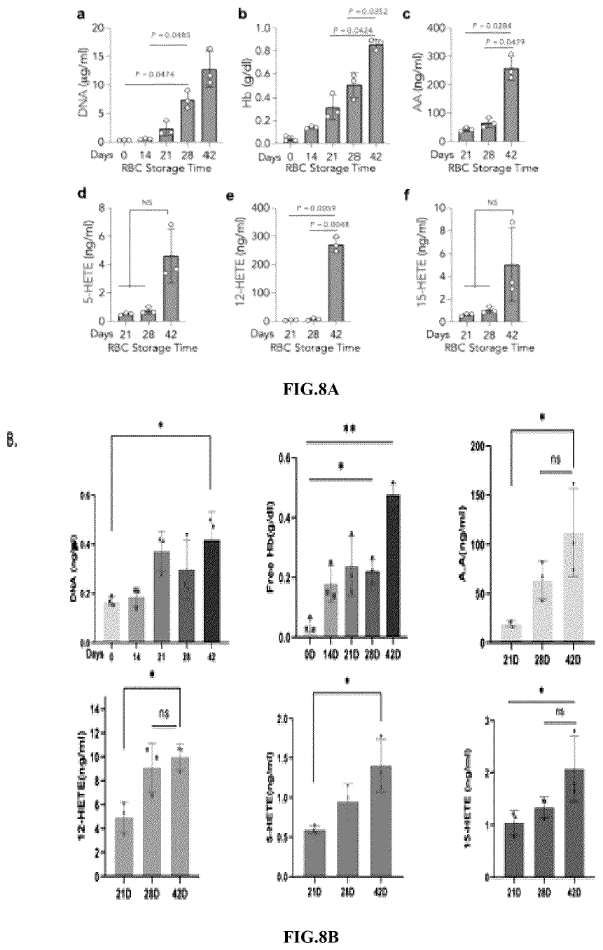

Additionally, these polymers did not leach from the scaffold (FIG. 10). 8 cm² nanofibre pAcr was weighed (6.75 mg) and incubated with 1 ml of distilled water for 30 min at ambient temperature. The incubated water was collected after 30 minutes and lyophilized. The lyophilized product was processed via fluorescence spectroscopy. pAcr was further subjected to sequential incubation with distilled water for 5 minutes. The results clearly show that no significant amount (pAcr) is leached during the treatment.

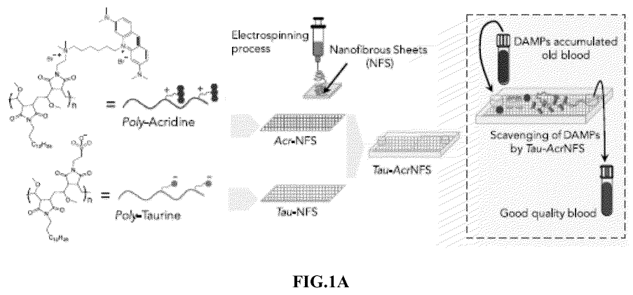

FIG. 2a revealed that a gradual increase in surface area (>8 cm²) and incubation time (>5 min) had no significant effect on the scavenging of extracellular DNA by pTau and pAcr scaffold groups. The idea of using cationic (pAcr) and anionic (pTau) scaffolds in combination for later scavenging experiments was based on the presence of a negative and positive charged lesion in pRBCs.

To ensure the effectiveness of scaffold towards positive and negative charged DAMPs, pTau and pAcr scaffolds of 12 cm² area each, (total surface area-24 cm²) were taken in combination to treat both NLR 42 D RBCs and supt. The cleaning of DAMPs from both the groups with scaffolds was performed for 5 minutes at 4° C. The whole mindset to design this experiment was to mimic the clinical scenario where RBCs units could be cleaned once with scaffolds during the storage timeline.

Firstly, the extracellular nucleosomes (DNA-histone complexes) were quantified via ELISA with and without the treatment. The findings were relatively plotted by considering nucleosome concentration as 100% for 42 D untreated NLR supt. The treatment of NLR 42 D supt with both the scaffolds resulted in reducing extracellular nucleosomes up to 50%. A similar scavenging efficiency was reported in the RBCs group (FIG. 2). The high percentage of extracellular nucleosomes in the NLR set indicates storage associated extracellular DNA-histone complex liberated from WBCs undergoing apoptosis, necrosis, or neutrophil extracellular traps NETosis (Zimrin, A. B. and Hess, J. R. Current issues relating to the transfusion of stored red blood cells. Vox Sang. 96, 93-103 (2009)).

Moving ahead, the extracellular DNA in NLR units reduced up to ˜75% in supt and ˜40% in the RBC group (FIG. 2). The fold decrease was significantly higher in the supt group compared to the RBCs treatment group. The difference in efficiency was due to the presence of RBCs shields the scaffolds that could hinder the direct interaction between scaffold and DNA.

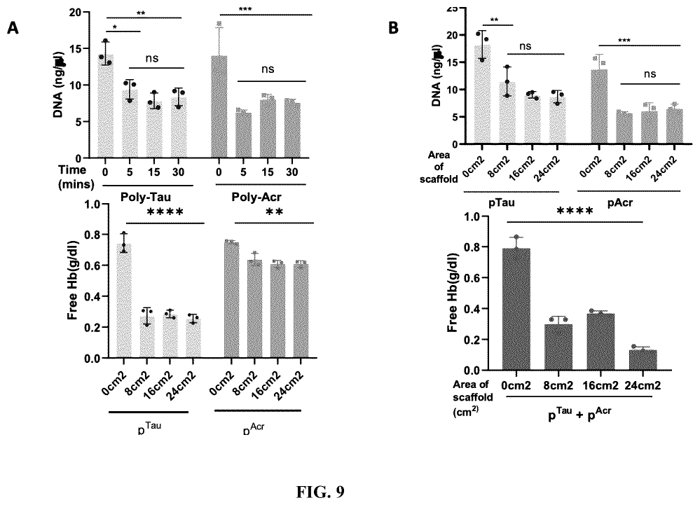

NLR supt was further subjected to free Hemoglobin (Hb) quantification, and the data unveiled a decrease in Free Hb concentration from ˜0.966 g/dl to 0.5 g/dl. This fall in Free Hb (g/dl) accounts for 50% scavenging ability of scaffold compared to the non-treatment group (FIG. 2). Significant scavenging was also observed when the RBCs unit was treated with scaffolds (FIG. 2). Interestingly, In LR units, the reduction in Free Hb was highly evident in the supt group (˜0.466 g/dl to ˜0.226 g/dl), but the RBCs treatment group (LR) did not show any significant drop (FIG. 11).

The universal role of scaffold to bind and scavenge DAMPs was further substantiated by LCMS findings. AA and 12-HETE were majorly scavenged in blood supernatant by the charged scaffolds. AA and 12-HETE drastically decreased from ˜234 ng/ml to ˜69.9 ng/ml and ˜270 ng/ml to ˜47 ng/ml, respectively (FIG. 2). A similar reduction was further recorded for 5 and 15-HETE. RBCs units also indicated a significant reduction, where scavenging was observed up to 70% for all the lipids. Compared to the findings of NLR, it was evident that LR blood had a low quantity of AA and other lipids. However, treatment with scaffold significantly reduced the AA value in LR supt from 115 ng/ml to 50 ng/ml, whereas in the RBCs units, it dropped to 35 ng/ml. Thus, charged scaffolds also provide a method to purify LR blood that contains soluble bioactive lipids generated from RBCs. These findings also substantiate that the —COOH group and hydrophobic chains in bioactive lipids bind specifically to charged hydrophobic scaffolds that aid their removal. Combined treatment with both the scaffolds (pTau and pAcr) showed remarkable scavenging of extracellular DNA, nucleosome, Hb and could prove to be a game-changer in blood transfusion research.

### Example 3: Intermittent Scavenging of DAMPs: 21^{st} and 28^{th} Day Treatment During Storage Enhanced Quality of Stored Old RBCs

The storage lesion is a denomination that generally includes all changes that occur as RBCs age while in storage solution. They can be classified as “young” (<14-21 days) and “old.” (>21 D) RBC units, based on the concentration of generated DAMPs (Hanna, V. S. and Hafez, E. A. A. Synopsis of arachidonic acid metabolism: A review. J. Adv. Res. 11, 23-32 (2018)). The kinetics data (FIG. 8) showed a significant increase of DAMPs during 21^{st} to 42^{nd} Day of storage. This observation triggered to envisage that the production of DAMPs may accelerate the deterioration of remaining healthy RBCs in the stored unit. It was hypothesized that reducing DAMPs from the blood unit at a particular point in the storage timeline (0-42 D) may enhance healthier RBCs at 42^{nd} Day of storage.

To identify that distinct point in the storage timeline, the kinetics data of DAMPs production in stored RBCs was revisited (FIG. 8). The findings revealed that the production of DAMPs was rapid during the storage window of 21^{st}-42^{nd} Day. The RBC quality was also observed to gradually deteriorate in the same time frame. Therefore, the 21^{st} and 28^{th} D of stored blood units (NLR and LR) were strategically chosen for the treatment. The blood units were treated with 24 cm² of charged nanofibre scaffolds (12 cm² each of pTau and pAcr) for 5 min at 4° C. and stored till the 42^{nd} day post collection. The DAMPs (extracellular DNA, nucleosomes, free Hb, bioactive lipids) were quantified at 42^{nd} Day of storage following intermittent treatment (21^{st} Day and 28^{th} Day).

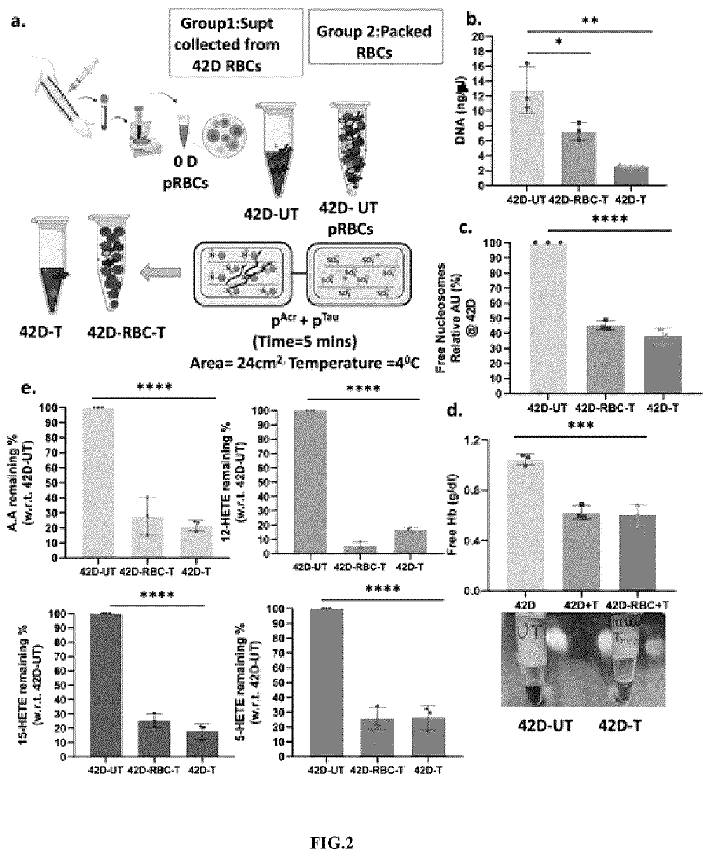

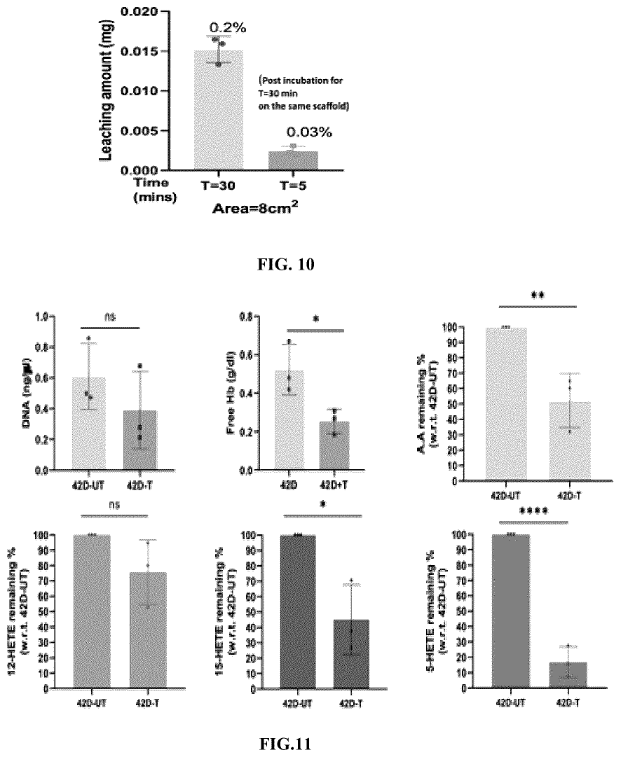

The production of extracellular DNA reduced remarkably from ˜12.7 ng/μl to ˜8.32 ng/μl on 42 D following a 21^{st} day treatment, while it was further reduced to ˜3.26 ng/μl if treated on the 28^{th} Day (FIG. 3). This reduction was in reference to the untreated 42 D NLR blood units. The production of extracellular DNA in LR blood units was low, as expected by the end of 42^{nd} D. Intermittent treatment on 21^{st} D did not have any significant decrease in its concentration, but 28^{th} D treatment showed a reduction up to 40% (˜0.3 ng/μl to ˜0.186 ng/μl (FIG. 12).

Nucleosome data revealed that 21^{st} D treatment was not adequate to limit its production at 42^{nd} D. However, the 28^{th} D treatment significantly decreased the generation of free nucleosome by approximately 60%. (FIG. 3). Thus, the data suggest that 21^{st} day cleaning might be a bit early to remove DAMPs as the chances of free nucleosomes production are higher in the remaining storage period, i.e., the next 21 D. The 28^{th} D intermediate treatment in LR units was also effective. It significantly reduced the production of free nucleosomes by 30%, although they were present in much lower quantities when compared to NLR (FIG. 12). Free Nucleosome quantification was followed by monitoring the levels of free hemoglobin. The 21^{st} D and 28^{th} intermediate treatment in NLR units revealed a similar reduction in the levels of free Hb, i.e., almost 50% (˜0.992 g/dl to 0.6 g/dl and 0.5 g/dl, respectively). However, the 21^{st} D treatment did not impact the lysis rate of RBCs in LR units whereas the 28^{th} D treatment limited the free Hb production by 50%, compared to the untreated group (˜0.4 g/dl to ˜0.2 g/dl). The efficacy of intermediate treatment was evident with quantifying free nucleosomes and free Hb in NLR and LR units.

However, it was further strengthened with the evaluation of bioactive lipids. The abundant AA in NLR units reduced to ˜32 ng/ml and ˜34 ng/ml when treated on 21^{st} and 28^{th} D compared to the untreated group (˜258 ng/ml). The effect of intermediate treatment was more evident in the case of 12-HETE production in the pRBCs unit. A 100-fold reduction (˜200 ng/ml to ˜2 ng/ml) was found in the levels of 12-HETE following a 21^{st}/28^{th} D treatment (FIG. 3). This states that the lesser production of 12-HETE, an oxidized AA product, is highly reliant on low AA formation in stored RBCs due to intermittent treatment. A similar trend was seen for 5 and 15-HETE. In LR stored units, there was a significant production of AA but intermittent treatment on 21^{st} D/28^{th} D drastically decreased its production by ten folds (FIG. 12).

This experiment suggested that intermediate treatment on the 21^{st} and 28^{th} Day leads to a significant reduction in the production of a few DAMPs at 42 D. However, 28^{th} day treatment was more desirable as it outscored the reduction of DAMPs following a 21^{st} day treatment. This strategy is expected to enhance the overall quality of the stored blood. Intermittent treatment could provide a significant breakthrough in transfusion-related complications. Stored blood with 28^{th} day intermediate treatment is expected to enhance the quality of stored blood and drastically bring down the transfusion-related complexities.

### Example 4: Intermittent Treatment with Scaffolds Maintains the Structural Integrity of RBCs at 42 D

Maintaining the structural integrity of pRBCs after treatment: It is estimated by the conventional osmotic fragility assay. The stored RBC units (treated and untreated group) were added to 9 tubes containing saline (NaCl; pH 7.4). The NaCl concentration gradually increased in each tube by 0.1%. The lowest being 0% (distilled water) (Tube 1) and the highest being 0.9% (Tube 9). The tubes were gently mixed and incubated at room temperature for 30 minutes. The incubation was followed by centrifugation at 500 G for 20 min, and supernatants were collected. The optical density of the supernatant was measured by Multi-Mode Microplate Reader (Thermo fisher, Massachusetts, USA) at 540 nm. Hemolysis in each tube was expressed in percentage. The maximum absorbance value of haemolyzed RBCs was taken 100% in the distilled water. The erythrocytes treated with normal saline were used as a negative control (0% hemolysis).

Primary and secondary fixation of RBCs for SEM: Glass coverslips were sonicated in acetone and cleaned. They were further dipped in 2% APTMS (3-(Aminopropyl) trimethoxy silane), diluted in acetone to impart a coating of APTMS. RBCs were primarily fixed with 2% glutaraldehyde (GTA), incubated for 30 min in dark, and washed with PBS. The primary fixed RBCs were incubated on APTMS coated coverslips for 1 h. After 1 h, the coverslips were rinsed twice with PBS and incubated with 0.1M cacodylate buffer for 10 min. Secondary fixation was achieved via 1% (w/v) Osmium tetroxide (OsO4) in 10 min. The secondary fixed coverslips were then rinsed with MilliQ water (10 min), followed by a dehydration step that involves a gradient ethanol wash, ranging from 40% to 100%. The dehydration step is followed by HMDS (Hexamethyldisilane) drying. Finally, dried samples were mounted on aluminium stubs, sputter-coated with gold, and imaged under FESEM (Carl Zeiss MERLIN VP compact).

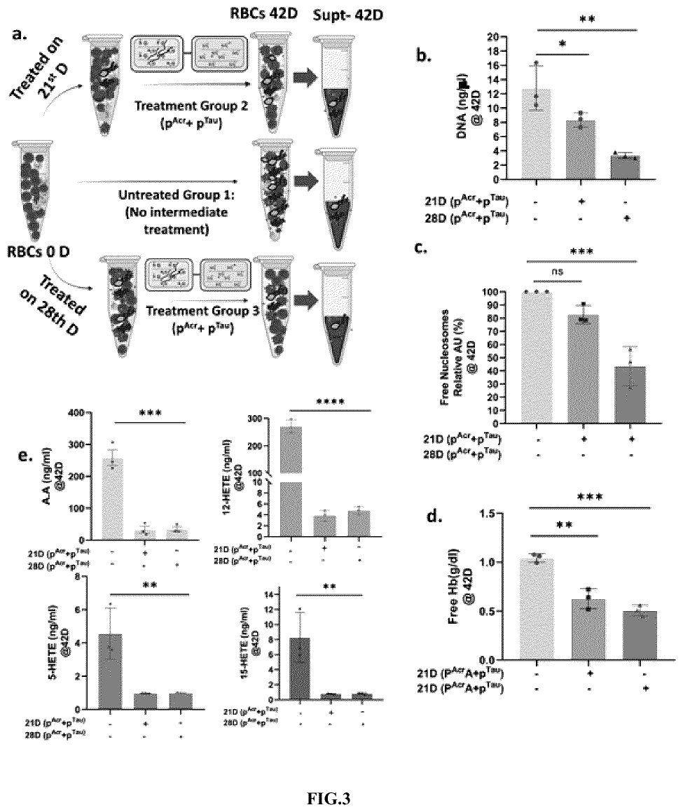

Discussion: The morphology of human red blood cells (RBCs) deteriorates progressively throughout hypothermic storage. Echinocytosis is the prevalent pathway that leads to these morphological deformities. As a result, each unit of stored blood contains a heterogeneous mixture of cells (discocytes, sphero-echinocytes, and spherocytes) at various stages of echinocytosis. Discocytes are regarded as the healthy RBCs (Fu, X., Felcyn, J. R., Odem-Davis, K. and Zimring, J. C. Bioactive lipids accumulate in stored red blood cells despite leukoreduction: a targeted metabolomics study. Transfusion 56, 2560-2570 (2016)). The morphology determines the health of RBC or its fate during circulation post-transfusion. Fresh RBCs (0 D) AND 42 D NLR stored untreated were imaged via FESEM to estimate the number of discocytes present at the end of the storage period. The images revealed that the major chunk of RBCs in 42 D NLR-pRBCs was either sphero-echinocytes or spherocytes. The healthy discocytes were negligible (FIG. 3). The observable discocytes from multiple 42 D samples were counted and considered 100%. The counted discocytes in 42 D untreated sets were taken as a reference to analyze the intermediate treatment groups (21^{st} and 28^{th}-day treatment). The image analysis revealed two folds higher discocytes in groups that received pAcr and pTau treatments on the 21^{st} D, whereas the 28^{th} D treatment leads to 3.5 folds higher discocytes population (FIG. 4). The higher number of discocytes in the treatment groups adds further evidence to the benefits of intermittent treatment, especially on the 28^{th} Day of storage by engineered scaffolds that helps in maintaining the excellent quality of RBCs.

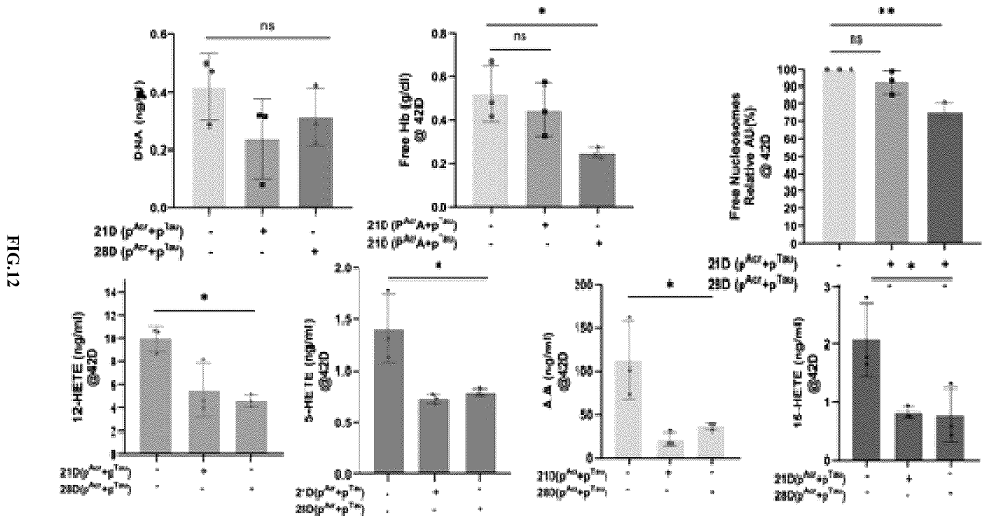

Digging more into stored RBCs quality, the Osmotic fragility test (OFT) is also a crucial parameter in the characterization of erythrocyte membrane health. It depends on the degree of resistance of red blood cells (RBC) to lysis when introduced in a hypotonic environment. OFT is also determined by 50% of the hemolysis at a specific NaCl concentration (%) known as MCF (Mean cell fragility). The value of MCF rises with the increase in the storage period of the pRBCs unit. In NLR pRBCs units, a steep increase in MCF value was observed where it increases from approx. 0.35 on 0 D to 0.6 on 42 D. The increase in value shifts the hemolysis curve to the right side (FIG. 4). This also compliments the FESEM data where 42 D RBCs population were majorly in the form of spheroechinocytes which gets more fragile and having low osmotic resistance. A pRBCs unit having a higher MCF value may lead to intravascular hemolysis after transfusion, which causes a reduction of the RBC life span in circulation. Surprisingly, the LR units also showed an increase in MCF values from approx. 0.35 to 0.5 stating the extent of RBCs health damage (FIG. 13). However, it is not as significant as NLR data, but it suggests that LR RBCs also get fragile during storage.

Shedding light on the intermittent treatment given to NLR and LR units on 21^{st}/28^{th} D of storage with both the scaffolds (pTau and pAcr), area 24 cm² for 5 minutes at 4° C. The data suggest that 21^{st} D intermittent treatment has significantly reduced the MCF value to approximately 0.52, whereas 28^{th} D intermediate treatment led the MCF of 42 D RBCs to coincide with the 28^{th} D stored unit (FIG. 4). The 21st and 28th D intermittent treatment in LR units reduced the MCF values from the untreated group, shifting the hemolysis curve to the left side, i.e., the healthy side. The reduction in MCF values suggests that RBCs were healthier after intermediate treatment and are more resistant to lysis. Out of both the time windows chosen for cleaning, 28^{th}-day treatment led to a significant reduction of DAMPs on the 42^{nd} Day. This clearly indicates that 28^{th} day cleansing is the perfect time to retain healthier RBCs.

### Example 5: Intermittent Scavenging of DAMPs from Stored Murine Blood Slows Down the Ageing that Displays a Way of Enhancing Shelf Life

### In Vivo Experiment Design: Mice

Both male and female mice of age 8-12-weeks-old were used for the experiments. The animals were bred and housed in the animal facility at the National Centre for Biological Sciences, Bengaluru. Animals were caged (maximum, four per cage) during the experiment, food and water were offered as libitum. All mice study strictly adhered to institutional and national guidelines for humane animal use. The experimental protocols were approved by the Institutional Animal Ethics Committee (IAEC) at the Institute for Stem Cell Science and Regenerative Medicine.

Packaging and Storage of Mice RBCs and their Treatment with pTau and pAcr to Scavenge DAMPs:

Blood collected in CPD was immediately transferred and centrifuged at 500 g for 20 min at 4° C. Plasma was carefully aspirated, and the pRBCs were pooled together. Optisol, also known as AS-5, was added to the pRBCs at 65% hematocrit. 550 uL and 250 uL blood units were finally aliquoted from these pRBCs and stored in sterile Eppendorf tubes (0.6 and 0.3 ml). Mice pRBCs were treated twice in combination with pTau and pAcr scaffolds (24 cm²) for 5 min on the 5^{th} and 10^{th} Day of storage at 40° C. After the scavenging procedure, the RBCs were again transferred in autoclaved 2 ml Eppendorf tubes, leaving a small residual space, and stored till the 17^{th} Day, and its supernatant was taken for extracellular Hb and nucleosome estimation.

Alleviate Exposure of Labelled Phosphatidylserine by Annexin V (AV) in Treated pRBCs:

The number of erythrocytes expressing phosphatidylserine (PS) on their outer membrane was determined as described elsewhere (Han, V., Serrano, K. and Devine, D. V. A comparative study of common techniques used to measure haemolysis in stored red cell concentrates. Vox Sang. 98, 116-123 (2010)). Briefly, labelling with AV was performed by adding 5 μL of AV-Alexa Fluor™ 568 conjugate (Invitrogen, USA) in 106 erythrocytes in 100 μL Annexin binding buffer. The binding buffer was prepared by adding 140 mM and 2.5 mM CaCl2 in 10 mM HEPES Buffer (all from Sigma-Aldrich). After incubation on ice for 15 minutes, cells were diluted four times with binding buffer and analyzed on an LSR Fortessa cytometer. Data analysis was performed with computer software (FACSDiva). The erythrocyte population with exposed PS labelled with AV-Alexa Fluor™ 568 conjugate was measured as a percentage of Annexin V-positive RBCs.

### Experimental Design for Biotinylation and RBC Transfusions:

EZ-Link Sulfo-NHS Biotin (Thermofisher Scientific, US) was used for IV injections to get biotinylated RBCs. This results in the covalent amide linkage between biotin and lysine residues of outward-facing RBC membrane proteins (Singer, V. L., Jones, L. J., Yue, S. T. and Haugland, R. P. Characterization of PicoGreen reagent and development of a fluorescence-based solution assay for double-stranded DNA quantitation. Anal. Biochem. 249, 228-238 (1997)). Briefly, sulpho-NHS biotin powder was dissolved in sterile PBS, vortexed, and passed through a 0.22-μm filter. It was diluted to a final concentration of 1 mg biotin per 300 μL 1×PBS. Mice were restrained, and the biotin reagent solution was administered through intravenous injection. Post-2 d of the biotin administration, under mild anaesthesia, around 1 ml of biotinylated blood was collected through a cardiac puncture in CPD containing vials and immediately centrifuged at 500 g for 20 min at 4° C. Plasma was carefully aspirated, and pRBCs were pooled together. The biotin-labelled blood was processed and stored using Optisol as a preservative in sterile Eppendorf tubes (0.5 and 1 ml).

Treatment of Biotin-labelled RBCs with pTau and pAcr scaffolds: Functionality Tests: Biotin-labelled RBCs was divided into treated and untreated groups. The treatment group (n=3) pRBCs units were cleaned with scaffolds on the 5^{th} and 10^{th} Day of storage and then packed until 18 D. The untreated group pRBCs units (n=3) were not treated with scaffolds during the storage timeline. Both the groups were analyzed for labelling efficiency and phosphatidylserine exposure during the storage.

Evaluating the efficiency of Biotin-labeled RBCs and their enhanced shelf life by recovery experiment: At different time points during the storage (0 D, 7 D, 14 D, 15 D and 17 D), 6 μl of RBCs from both the groups (treated and untreated, n=3) was conjugated with Streptavidin APC-efluor® 780 Conjugate (BD biosciences, USA). The positive RBCs population labelled with streptavidin APC-efluor was processed and analyzed via LSR Fortessa cytometer (BD Bioscience, San Hose, CA). On different days of storage (0 D, 7 D, 14 D, 15-19 D), biotinylated pRBC units with and without treatment were diluted (1:1) with PBS. 200 μl of diluted biotinylated pRBC was administered to the restrained mice via intravenous injection (Burger, P., De Korte, D., Van Den Berg, T. K. and Van Bruggen, R. CD47 in erythrocyte ageing and clearance—the dutch point of view. Transfus. Med. Hemotherapy 39, 348-352 (2012)). At specific time points, 6 μl of blood was collected via tail snip and stored in microfuge tubes containing 250 μL of sterile PBS.

To detect biotinylated RBCs through flow cytometry, approximately 10⁶-10⁸ RBCs (100 μL of the diluted sample) were added to 0.125 μg of streptavidin-APC-efluor® 780 Conjugate (1:20 of 0.2 mg/mL; eBioscience™). Samples were incubated in the dark for 5 min, washed with 400 μL PBS, and centrifuged at 1000 g for 4 min. The RBC pellet was resuspended in 200 μL PBS. Samples were analyzed using BD LSR Fortessa cytometer (BD biosciences, USA). The red laser was used to excite the dye (λ emission=780 nm) with a bandpass filter of 780/60. The following formulae calculate the post-transfusion survival (%).

$$\frac{Post transfusion RBC recovery ( \% ) at t ( t )}{Post transfusion Rbc Recovery at t ( 1 h )} \times 100$$

where (t) stands for post-transfusion time point, i.e., 1 h, 24 h, 48 h, and 72 h. This formula is used for all pRBCs units stored for different days and then transfused in a different set of mice. The data were analyzed with FACS Diva Software (BD Biosciences).

### Statistical Analysis:

All data are shown as mean±SD (standard deviation). Data were plotted and analyzed using Prism software. One-way ANOVA with Tukey multiple comparison test (*P<0.05, **P<0.01, ****P<0.0001) were performed for comparisons between multiple groups.

Discussion: Designing a functionality experiment for Human RBCs after transfusion was not ethically feasible, leading to its scrutinizing via murine model. The shelf life of C57 BL/6 mice RBCs is reported to be 14 days which is equivalent to human RBCs (42 D) (Piety, N. Z., Reinhart, W. H., Pourreau, P. H., Abidi, R. and Shevkoplyas, S. S. Shape matters: the effect of red blood cell shape on perfusion of an artificial microvascular network. Transfusion 56, 844-851 (2016)). The production of DAMPs was estimated in the mice RBC units to identify the potential window for intermittent cleaning. The mice blood was drawn via retro-orbital puncture and processed as described in previous sections. The stored RBCs unit was then assessed for the production of DAMPs for 14 D. The data suggested a significant production of free Hb during 10 D-14 D of storage. The levels of free Hb increased from ˜0.2 g/dl to˜ 0.4 g/dl. A two-fold increase was also observed in the production of free nucleosomes. These observations suggested that mice-stored RBCs also display a similar formation of DAMPs as in human blood.

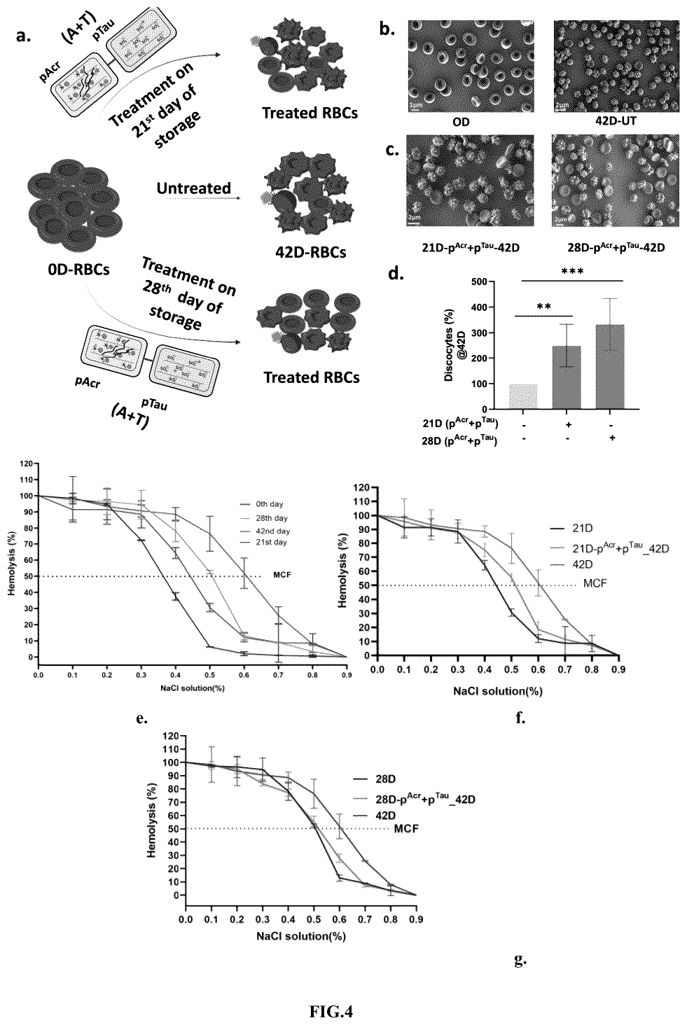

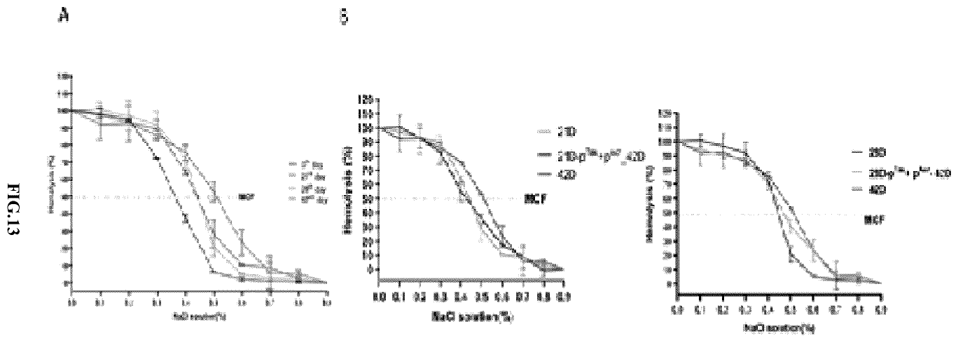

Since the production of DAMPs was significant between the 10^{th} and 14^{th} Day, a 5^{th} and 10^{th}-day intermediate treatment was chosen to evaluate its effect on stored mice blood. The treatment was performed on either the 5^{th} or 10^{th} D using 24 cm² scaffolds (pTau and pAcr) for 5 minutes at 4° C. To further enhance our understanding of the intermediate treatments, another experimental group was added to this study. The mice blood in this group was treated on the 5^{th} and 10^{th} Day, i.e., the blood was treated twice (dual treatment). The evaluation of DAMPs in 14 D blood supt post single/twice treatment revealed that dual treatment overpowered the single one. The single treatment had lessened the formation of free Hb significantly, but the dual treatment reduced the free Hb levels to ˜0.15 g/dl (FIG. 5 and FIG. 14). The dual treatment also resulted in a lower production of nucleosomes (˜30%) when compared to the untreated group. These findings laid a foundation for future experiments where an increased number of intermittent treatments could lead to a better RBC quality than a single treatment. The shelf life of RBCs is inversely proportional to the production of DAMPs. The generation of DAMPs due to the biochemical changes in RBC accelerates its deterioration. Thus, scavenging of DAMPs improves the quality of the stored blood and has the potential to enhance its shelf life. The intermediate treatment with scaffolds opened new gateways to enhance the shelf life of murine RBCs.

Further, to understand the shelf-life of RBCs, the phospholipid architecture of RBCs was studied. Storage-associated loss of phospholipid asymmetry may result in phosphatidylserine (PS) exposure in the outer leaflet of RBC's plasma membrane. This serves as an effective signal contributing to the recognition and phagocytosis of apoptotic cells and cell fragments. PS exposure has been implicated in the removal of damaged RBCs from the circulation. It is well established that PS exposure increases with storage and is linked with DAMPs production (Hod, E. A. et al. Transfusion of red blood cells after prolonged storage produces harmful effects that are mediated by iron and inflammation. Blood 115, 4284-4292 (2010)). Increased PS exposure during storage is also associated with hemolysis and microvesicles concentration in red blood cell concentrates. This Ultimately leads to the low survivability of transfused RBCs in circulation. With this background information, the idea was to examine PS exposure (%) in the outer leaflet of RBCs membrane throughout storage time. The findings suggest that in the untreated samples' PS exposure (%) was low till the 5th Day (˜5-6%). However, it increased to ˜20% by the end of the 14^{th}D (FIG. 5) and shoots up to two folds (˜40-50%) within three days, i.e., 17^{th} Day. The treatment group was contacted with scaffold on 5^{th} and 10^{th} D (twice treatment). The treatment group revealed a marked decrease in the PS exposure (%) on the 14 D (˜10%). Interestingly, the PS exposure (%) was significantly low on the 17 D (˜18%) when compared to the untreated (NT) group (FIG. 5). The low exposure (%) of PS in the treated group indicates the preservation of RBCs' phospholipid architecture until 17^{th} D. It also provides solid evidence that removing DAMPs from the stored blood via twice treatment (5^{th} and 10^{th} Day) slows down the senescence or ageing of RBCs.

To further supplement this claim, biotin was transfused in both male and female mice (8-12 weeks). The sulfo-NHS-biotin binds covalently and labels membrane proteins with Biotin (Dinkla, S. et al. Phosphatidylserine exposure on stored red blood cells as a parameter for donor-dependent variation in product quality. Blood Transfus. 12, 204-209 (2014)). After 24 hrs, RBCs were collected from the same set of mice, processed, and stored as specified previously. The PS exposure data cemented that treated RBC units have their membrane architecture intact until 17^{th} D, i.e., three days more than the reported shelf life (14 D).

This understanding led to the storage of biotin-labeled RBCs units up to 19 D with and without treatment. Here, the twice intermittent treatment was provided as stated before. It is believed that, this is the first time that labeled RBCs were stored for more than two weeks. A few μl from different storage times was taken from the treated and untreated groups, and the percentage of the labeled RBCs was quantified using flow cytometry. The statistics revealed that RBCs' membrane integrity (˜85%) was reserved for 14 D in the untreated group. However, the membrane integrity significantly fell post 14^{th} Day. It was around (˜25-40%) for the 17^{th}-day stored blood (FIG. 5). The decline in the percentage of labeled murine RBCs affirms the loss in their membrane architecture. The RBCs incur morphological changes during storage that are evident after the storage exceeds the shelf life, i.e., 14 D.

On the contrary, the membrane integrity was intact (˜ 80-85%) until 17^{th} D in the treatment group. This suggests that RBCs membrane architecture could be preserved via intermediate scaffold treatment (5^{th} and 10^{th} Day). However, the labelling efficiency declined after 17^{th} D, indicating the storage limit of treated RBCs.

### Example 6: Twice Treated 17 D Murine RBCs had Reduced Dose-Responsive Pro-Inflammatory Cytokine Responses and Increased RBC Survival Post-Transfusion

Reduction in pro-inflammatory cytokine levels by transfusing treated pRBCs: Mice (age 8-12 weeks (males) bred in-house at inStem/NCBS animal house) were infused with RBCs stored for 14 and 17 D respectively, with and without pTau and pAcr treatments. Treatment and non-treatment groups were made as mentioned in Section 8a. After 2 hours of transfusion, Mice Plasma collected and analyzed for pro-inflammatory markers at 1:10 dilution with array buffer. Cytokines/chemokines, including Complement Component 5A(C5A), Intercellular adhesion molecule-1 (ICAM-1), monocyte chemoattractant protein-1 (MCP-1), Stromal cell-derived factor1 (CXCL12), macrophage inhibitory protein-1 (MIP-1), keratinocyte-derived chemokine/CXCL1 (KC/CXCL1), Interleukin-16 (IL-16), TIMP Metallopeptidase Inhibitor-1 (TIMP-1), Interferon-gamma-induced protein 10 (CXCL-10), Interleukin-1 receptor antagonist (IL-1F3) and Granulocyte-colony-stimulating factor (G-CSF) were quantified using Mouse Cytokine Array Panel A (R&D Systems, Minnesota, USA) following the manufacturer's instruction.

Quantification of total iron in the spleen: Sample digestion and preparation. Splenectomy was performed in mice after 2 h of transfusion. Spleen from all the groups was taken in a tared 2 ml Eppendorf, and the dry weight was recorded. Then, 1 ml protein precipitation solution and 1 ml (8%) nitric acid were added to the spleen tube and homogenized for 2 min. The protein precipitation solution was prepared with 0.53N HCl and 5.3% trichloroacetic acid in HPLC water. The solution containing a homogenized spleen with a closed lid was boiled at 2000 C for 30 min. Cooled in RT for 2 min, and caps were opened to release air bubbles. Finally, the solution was centrifuged at full speed for 10 min. For total iron quantification, the supernatant from all the groups (n=3) was taken.

ICP-MS method. The optimized operating conditions used for ICP-MS (Shimadzu-2030) are summarized in Supplementary Table 2. A peristaltic pump was used to deliver the samples (0.1 mL/min) to the nebulizer, converting the sample into a spraymist using argon (Ar) gas. Automated adjustments were made for torch alignment, detector voltage, and ion lens voltages for optimized resolution, sensitivity, and stability across a broad range of atomic masses. The doubly charged ion/charge ratio (Ce2+ 69.95 to Ce 139.90) and oxide ratio (CeO+ 155.90 to Ce139.90) were also monitored and were maintained below 3% and 2.5% (intensity), respectively. The Prep Fast system was interfaced with the ICP-MS and auto-diluted the stock solutions to generate the calibration curves and QCs samples. The 30-ppb Indium (In) solution was also mixed in-line by the Prep-Fast during the analysis as an internal standard to all the analyzed samples and standards. The method was evaluated for its selectivity, sensitivity, linearity, precision, and accuracy.

TABLE 2
The optimized operating conditions used for ICP-MS.

| Parameter | Settings |
|---|---|
| RF-Power (W) | 1200 |
| Carrier gas flow (l/min) | 1.2 |
| Plasma gas flow (l/min) | 15 |
| Auxiliary gas flow (l/min) | 1.0 |
| Spray chamber | Water cooled double pass |
| Spray chamber temperature (° C.) | 5.0 |
| Lens voltage | 4.5 |
| Mass resolution | 0.8 |
| Integration time points/ms | 3 |
| Points per peak | 3 |
| Replicates | 3 |

Statistical analysis: In experiments with multiple groups, ordinary one-way ANOVA with Tukey's post hoc test was used. In experiments with multiple groups, which are time-course studies, repeated measures of one-way ANOVA were used. The two-tailed Student's t-test with Welch's corrections was used to compare two experimental groups. The probability value (P)<0.05 was considered as a statistically significant difference. Statistical analysis and graphing were performed with GraphPad PRISM9.

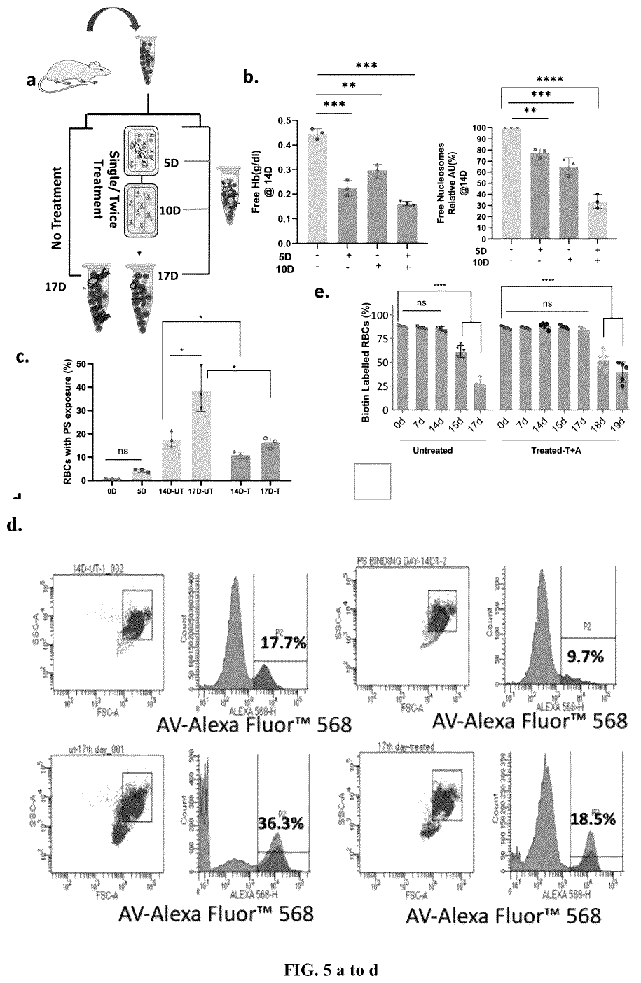

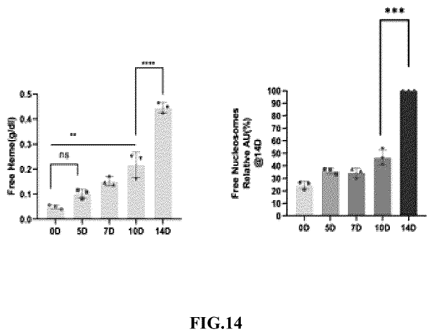

Discussion: To determine inflammation associated with the transfusion of treated and untreated, RBCs of 14^{th} and 17^{th} D were transfused in C56B6/J mice (male and females). The treatment condition was the same as mentioned earlier. 2 hours-post-transfusion, the mice were sacrificed, and the cytokines levels were measured in plasma.17 D untreated stored RBCs recorded elevated cytokine levels, i.e., two-fold higher than the treated 17 D group. The 17 D NT group had elevated expression of C5A, CXCL12, M-CSF, KC/CXCL1 (FIG. 6). In the same group (17 D untreated), increased IL-16, TIMP-1, CXCL-10, IL-1F3, G-CSF were also identified. However, this set of cytokines was not observed in the 14 D old untreated group, strengthening our earlier claim that the deterioration rate increases rapidly once RBCs past their shelf life. (FIG. 6). In the 17 D treated set, the cytokines levels were significantly low, and few pro-inflammatory cytokines were not even detected (FIG. 15). In addition, the spleen of both treated and untreated groups was harvested, and darkening of the spleen was seen at necropsy in mice transfused with 17 D untreated RBCs.

The observation of darkened spleen is highly associated with free iron deposition in the spleen after transfusing bad quality blood loaded with lysed RBCs and free Hb. On the other side, treated 17 D blood did not manifest darkening of the spleen that eventually affirms the RBCs were in good health even after crossing their shelf life (14 D) (FIG. 6). The group's spleen was taken and processed for free Fe²⁺ quantification through ICP spectroscopy to uphold our claim. The study altogether forms the cornerstone that twice intermittent treatment with charged scaffolds (pTau and pAcr) inhibits inflammatory response, enhances RBCs quality, and delays the RBCs senescence.

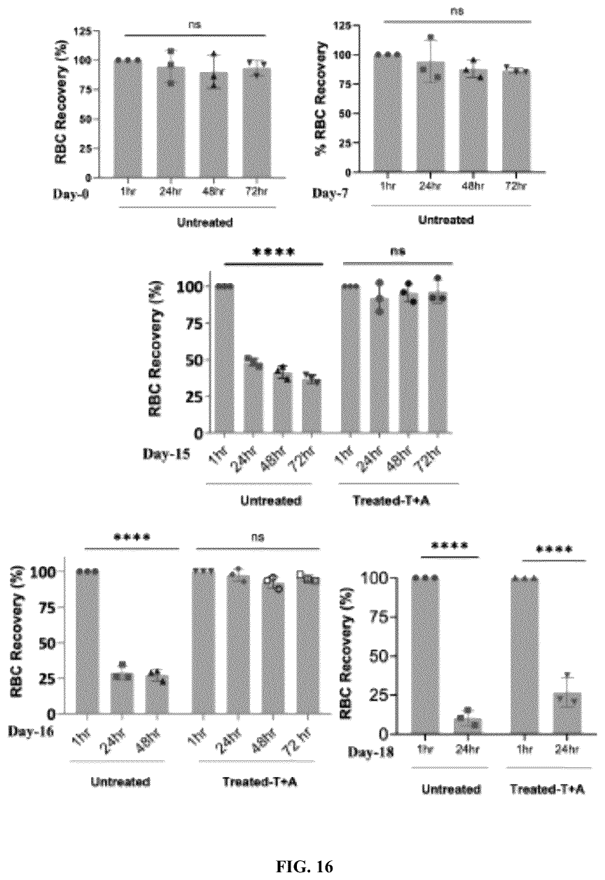

Subsequently, the stored red cell functionality was investigated through the survival of biotin-labeled RBCs in circulation in male/female mice (8-12 weeks) after transfusion. According to the FDA, the successful transfusion is established based on posttransfusion RBC survival of 75% or higher after 24 h (Mock, D. M. et al. Development, validation, and potential applications of biotinylated red blood cells for posttransfusion kinetics and other physiological studies: evidenced-based analysis and recommendations. Transfusion 58, 2068-2081 (2018)). Monitoring the rate of successful pRBCs transfusion in our studies, biotin-labeled RBCs stored for up to 19 D with and without treatment were transfused in a new set of mice. The labeled RBCs were tracked for their survival post-transfusion after 1, 24, 48, and 72 h via flow cytometry. To begin with, 0 D and 7 D old pRBCs transfusion without treatment were quantified for its survival up to 72 h. Since transfused labeled RBCs get mixed with the non-labeled RBC of recipient mice, (%) labeled population obtained post 1 h transfusion is considered as 100% that is circulating in recipient mice. The 0 D and 7 D RBCs recovery data revealed >75% RBC's survival in circulation after 24 h and 72 h of post-transfusion (FIG. 17).

As anticipated, transfusing 14 D stored NT RBCs in recipient mice showed more than 75% recovery after 24 h. When the time points were extended up to 72 hrs, a noticeable drop of labeled RBCs was observed after 48 h, and the survival population was reduced to 50% (FIG. 6). As per the FDA guidelines, the survival studies have been performed within 24 h which is the “gold standard” criteria. Thus, our study tweaks the current experimental settings and shown the labeled RBCs survival up to 72 h. The post 24 h recovery data gives a novel insight into the consequence of RBCs quality that plays a pivotal role in its function and survival in circulation. Contrarily, twice intermediate treated 14 D stored RBCs with scaffolds showed RBC survival of more than 75% for three days (FIG. 6). The 48 h-72 h recovery (%) sets prove that the transfused RBCs were in good health. This also effectively asserts that intermittent treatment slows down the ageing of stored RBCs and circumvents their clearance from mice circulation by spleen.

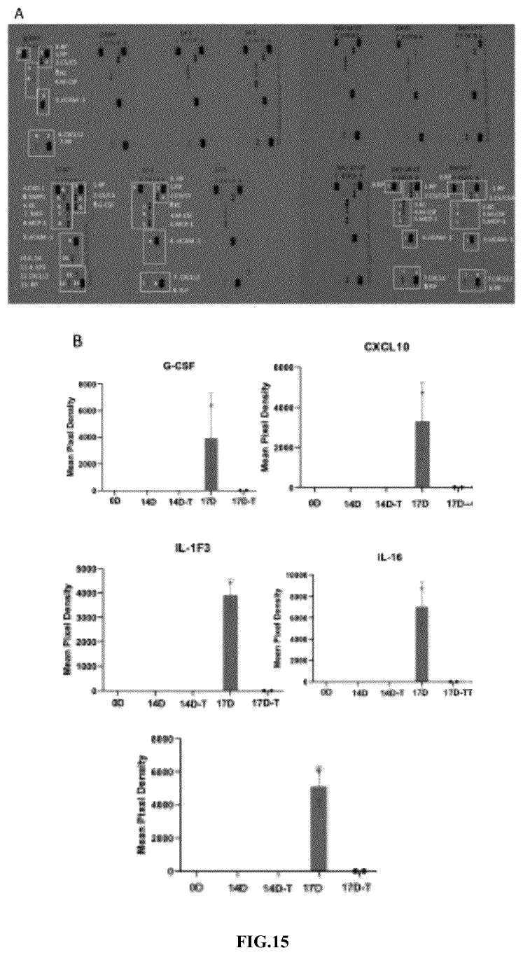

When 15 D untreated RBCs were transfused in mice, the survival (%) significantly decreased to 50%, which was entirely predictable (FIG. 6). A similar trend was observed for 15 D, 16 D and 17 D stored RBCs without scaffold treatment as they are instantly removed from circulation. Their recovery post-transfusion was also lower than 75% (FIG. 4). The Transfusion of 15 D old treated RBCs leads to 85% survival post 24 h, 48 and 72 h (FIG. 6). 16 D old RBCs unit were also in circulation for 72 h in recipient mice after transfusion in the treatment group (FIG. 16). However, the transfusion of 17 D old treated RBCs resulted in lower survival post 24 h. The survival declined to 50% at 48 and 72 h (FIG. 6).

The recovery data draws an equivalence between the 17 D treated RBCs and the to 14 D NT RBCs. In both groups, the transfused population was removed from circulation after 24 h. This equivalence states that intermittent treatment during the storage period can push the 14 D mice RBC's shelf life to 17 D. Three days of enhanced shelf life in murine is equivalent to nine days in human RBCs. The shelf life could not be pushed further as the recovery (%) in 18 D treated RBCs was below 75% (FIG. 16). This exclusive study on RBCs functionality adds up the new traits of scaffold treatment that can improve RBC's quality and prolong their shelf life.

Red blood cells (RBCs) transfusion is a life-saving process, and their stored units are the most globally demanded transfusion product. They are essentially required for symptomatic anemia, acute sickle cell crisis, acute blood loss of more than 30% of the blood volume, and restoring tissue oxygen delivery. However, the storage time influences the RBC units at a qualitative level. The RBCs age in the storage bag and develop storage lesions (DAMPs). These DAMPs (nucleosome, extracellular DNA, free Hb and bioactive lipids) were quantified in this study. The level of DAMPs reached its maximum by the end of 42 D, i.e., the shelf life of RBC. Therefore, transfusing RBC units with a hefty load of DAMPs could lead to transfusion-related complexities such as TRALI, deep vein thrombosis, and organ dysfunction (García-Roa, M. et al. Red blood cell storage time and transfusion: Current practice, concerns and future perspectives. Blood Transfus. 15, 222-231 (2017) and Martinod, K. and Wagner, D. D. Thrombosis: tangled up in NETs. Blood 123, 2768-2776 (2014)).

Currently, Leukoreduction (LR) decreases the production of DAMPs by eliminating white blood cells (WBCs) as the sole source of extracellular DNA, nucleosomes. However, it could not reduce bioactive lipids, free Hb or iron ions produced from RBCs. A variety of filter units are also available in the market that consist of a polymer as adsorbent and their membrane is decorated with engineered proteins that bind with pathogen toxins, i.e., PAMPs (Pathogen associated Molecular Pattern) (Roussel, C., Buffet, P. A. and Amireault, P. Measuring post-transfusion recovery and survival of red blood cells: Strengths and weaknesses of Chromium-51 labeling and alternative methods. Front. Med. 5, 1-8 (2018)). However, they fail to cover the whole spectrum of storage lesions associated with RBC.

Stored NLR units used for transfusion in clinical scenarios harbor whole spectra of charged lesions. It ranges from free histones, Hb Fe²⁺, DNA, and bioactive lipids (Rimmelé, T. and Kellum, J. A. Clinical review: Blood purification for sepsis. Crit. Care 15, 1-10 (2011)). The lack of a reported approach to eradicate these storage lesions incited us to design a technology to combat transfusion-related complexities. The present approach involves scavenging soluble DAMPs from old stored blood without disrupting the RBCs membrane. The hydrophobic cationic (pAcr) and anionic (pTau) polymers were engineered into charged nanofibre scaffolds. They bind via non-covalent interactions (electrostatic, π-π stacking, hydrophobic) to DAMPs present in old stored pRBC units. The experimental findings assert that a combination of pTau and pAcr scaffolds scavenges a wide range of charged lesions in 42 D NLR and LR groups (supt and RBC units). The DAMPs like extracellular DNA and nucleosomes were cleaned up to 50% in the 42 D NLR treated groups (supt and RBCs).

Furthermore, the scaffolds could scavenge 60-70% non-polar bioactive lipid and 40-50% free hemoglobin. These values validated the broad-spectrum cleaning of lesions by cationic and anionic scaffolds. The charged scaffolds were hemocompatible and did not leach into the aqueous media (RBC units). Post incubation with RBC units, The FESEM images depicted that the RBCs did not adhere to the scaffolds. This way, eliminating the DAMPs from the old blood units before the transfusion is expected to tackle transfusion-related complications. The cornerstone of this study is the intermittent treatment by scaffolds at the 21^{st} and 28^{th} D of RBCs storage. The resulting data showed that intermittent treatment leads significant reduction of DAMPs compared to untreated RBCs units on the 42^{nd} Day of storage. The results suggest that the 28^{th}-day treatment results in maximum reduction of DAMPs and healthier NLR RBCs on the 42^{nd} Day. This could be because the accelerated deterioration of RBCs majorly occurs between 28 D-42 D of storage, and in few cases, the significant formation was observed after 21 D itself.

The present study was not restricted to the NLR RBCs unit; the kinetic study of DAMPs production in LR units was investigated. Though their production rate was not significant like NLR units, the release of free hemoglobin and bioactive lipids raised interest. Thus, cleaning LR units before transfusion could significantly reduce the amount of RBCs associated with lesions. In this case, the concept of intermittent treatment worked efficiently where the free Hb and bioactive lipids concentration was significantly reduced when treated on the 21^{st} or 28^{th} or 42^{nd} D of storage.

The structural integrity of RBCs plays a vital role in increasing their residual circulation time. The osmotic fragility data supports that the scaffold-treated RBCs (28^{th}-day intermediate treatment) were healthier on the 42^{nd} Day of storage. Their membrane integrity was highly intact compared to the untreated RBCs.

It was envisaged that maintaining membrane integrity should lead to extended survival in circulation (enhanced functionality). In order to prove this hypothesis, the murine model was chosen for the functionality experiment. Provided, Murine RBCs have a shelf life of 14 D, the stored RBCs were intermittently treated twice on the 5^{th} and 10^{th} D of storage with pTau and pAcr scaffolds. The free nucleosomes and Hb after intermediate treatment were quantified on the 14^{th}-day blood supernatant. Compared to the untreated group, the drop in extracellular nucleosomes and DNA to 70% and 50% cements the pertinence of increasing the treatment numbers.

The biotin labeling experiment affirmed that scaffold treatment preserves the lipid symmetry of the plasma membrane. The data also proves that the shelf-life of RBCs could be pushed by 3 days. The recovery experiment presents a new standard to assess transfused RBCs' quality and functionality. The 14 D stored RBCs without treatment showed a recovery of >75%, 24 h post-transfusion. This was as per FDA standards. However, the recovery declined to post 24 h. This was attributed to their deteriorated condition, contributed by DAMPs formation. This phenomenon was not witnessed in the 14 D treatment group as labeled RBCs were in circulation for 72 h. The functionality of the 17 D old treated RBCs was also >75% post-transfusion and equivalent to untreated 14 D stored RBCs.

This suggests that scaffold treatment pushed the murine RBC's shelf life up to 3 days. This is equivalent to 9 days for human RBCs. Additionally, the pro-inflammatory cytokine levels were very low for the 17 D old treated RBCs post-transfusion, and there was no significant darkening of the spleen observed. Therefore, our study unfolds the link between the accelerated deterioration of RBCs caused by produced DAMPs. Gazing to the bright front, scaffolds treatment before the transfusion could significantly reduce the storage lesion and prevent transfusion-related complexities. Further, the intermittent treatment adds a new dimension to maintain RBC's quality.

The detailed investigation here explains the concept of scavenging DAMPs during storage that Ultimately leads to the increased shelf life of RBCs. This research not only has a massive potential to meet the enormous demand of RBC units but also addresses the transfusion-related complexities associated with old stored blood. While the present disclosure provides experimental data only for RBCs, the present invention could also be applied to other biological material and other stored cells like platelets and stem cells.

### Example 7: Zwitterionic Polymer Synthesis: Synthesis of Zwitterionic Polymer from PMVEMA

PMVEMA-C14(50%): The PMVEMA (500 mg, 3.2 mmol) was added to tetrahydrofuran (10 ml) in pressure tube and kept for stirring at 50 deg until dissolution. After dissolution, solution of tetradecyl amine (341 mg, 1.6 mmol) in THF (10 ml) was added to the PMVEMA solution and kept the reaction mixture for 3 h at 50 deg. The solvent was removed using rota vapor to get the polymer in dry powder form (800 mg, ˜90% yield).

PMVEMA-C14-Zwitr: A solution of zwitterionic compound, a sultone derivative of amine (134.59 mg, 0.6 mmol) was prepared in 10 ml DMF and added dropwise to the solution of PMVEMA-C14 (800 mg) in THF (20 ml) and left for 12-16 h with stirring at 80 deg. The reaction mixture was cooled down and the reaction mixture was precipitated in cooled ethyl acetate. The polymer was washed with water to remove salts. Then the solid product was lyophilized and white powder (510 mg) was observed. [FIG. 21]

Although, the foregoing description reveals general nature of the invention herein such that others can, by applying current knowledge, readily modify and/or adapt for various applications such specific description without departing from the generic concept, and, therefore, such adaptations and modifications should and are intended to be comprehended within the meaning and range of equivalents of the disclosed description. Furthermore, precise and systematic details on all above aspects are currently being made. Work is still underway on this invention. It is to be understood that the phraseology or terminology employed herein is for the purpose of description and not for limitation. Therefore, while the description herein is described in terms of preferred embodiments, those skilled in the art will recognize that the embodiments herein can be practiced with modification within the spirit and scope of the embodiments.

1. Nanofibre scaffold for selective scavenging of storage lesion from stored cells comprising a backbone polymer functionalized with a group selected from:
(i) an anionic group,
(ii) a cationic group,
(iii) a combination of anionic group and cationic group, or
(iv) a zwitterionic group.

2. (canceled)

3. (canceled)

4. The scaffold as claimed in claim 1, wherein the functionalized polymer is in electrospun form.

5. The scaffold as claimed in claim 1, wherein the backbone polymer is selected from the group comprising poly(acrylic acid), poly(4-vinylpyridine), Poly(4-vinylphenol), poly(allylamine), poly(vinyl alcohol), poly(methyl vinyl ether-alt-maleic anhydride) (PMVEMA), poly(vinyl chloride), poly(glycerol), poly(2-hydroxyethyl-methacrylamide), poly(2-((allyloxy)methyl)oxirane), poly(vinylpyrrolidone), poly(4-vinylbenzyl chloride-alt-maleic anhydride) and poly(vinylimidazole).

6. The scaffold as claimed in claim 1, wherein the backbone polymer is poly (methoxy vinyl ether-alt-maleic anhydride (PMVEMA).

7. The scaffold as claimed in claim 1, wherein the polymer is functionalized with an anionic group present in an amount from 8 to 50% and is selected from 1,3-propane sultone, taurine, 1,3-dioxolan-2-one, 1,2-oxathiane 2,2-dioxide, phosphorochloridic acid, (3-aminopropyl)phosphonic acid, Pentanoate, 2-(methylamino)ethyl hydrogen phosphate, pentyl hydrogen phosphate and 4-(butylamino)butyl sulphate, preferably taurine.

8. (canceled)

9. The scaffold as claimed in claim 1, wherein the polymer is functionalized with a cationic group present in an amount from 3 to 35% and is selected from ethidium bromide derivative, cyanine dye, YO-1, proflavine derivative, acridine orange, ethylene blue derivative, ellipticine derivative, phenanthridine derivative, quinolinium derivative, thalidomide derivative, naphthalimide derivatives and quinoline derivative, preferably acridine.

10. (canceled)

11. The scaffold as claimed in claim 1, wherein the cationic polymer has intercalating and positive charge characteristics.

12. The scaffold as claimed in claim 1, wherein the scaffold is in the form of a sheet, bead, bag, insert device, tube, and the like, preferably a nanofibrous sheet.

13. The scaffold as claimed in claim 1, wherein the stored cells are selected from red blood cells (RBCs), white blood cells (WBCs), platelets and stem cells, preferably red blood cells (RBCs).

14. A method for treating stored cells or scavenging damage-associated molecular platforms (DAMPs) from stored cells, wherein said stored cells are treated with the scaffold as claimed in claim 1.

15. (canceled)

16. A method of enhancing quality and shelf-life of stored cells or enhancing cell membrane integrity of stored cells by scavenging DAMPs from stored cells wherein said stored cells are treated with the scaffold as claimed in claim 1.

17. (canceled)

18. The method as claimed in claim 16, wherein the DAMPs are extracellular DNA, histones, nucleosomes, cell-free hemoglobin, iron, proteins, and polyunsaturated fatty acids.

19. (canceled)

20. A kit comprising scaffold as claimed in claim 1, and instructions for passing the stored cells through said scaffold.

21. (canceled)

22. A method for preparing a nanofibre scaffold as claimed in claim 1, comprising:
(a) preparing a solution of polymer functionalized with a group selected from an anionic group, cationic group or zwitterionic group; and
(b) electrospinning the solution to obtain a nanofibre scaffold.

23. The method as claimed in claim 23, wherein said polymer is made hydrophobic before functionalization with a group selected from an anionic group, cationic group or zwitterionic group.

24. A system for selective scavenging of storage lesion from stored cells wherein the system comprises a nanofibre scaffold as claimed in claim 1 having a backbone polymer functionalized with an anionic group and a scaffold having a backbone polymer functionalized with a cationic group or having a backbone polymer functionalized with a zwitterionic group, wherein the storage lesion are selectively scavenged when stored cells are placed in contact with said scaffolds.

25. The method as claimed in claim 14, wherein the DAMPs are extracellular DNA, histones, nucleosomes, cell-free hemoglobin, iron, proteins, and polyunsaturated fatty acids
